(19) United States
(12) Patent Application Publication
(10) Pub. No.: US 2006/0241177 A1
(43) Pub. Date: Oct. 26, 2006

(54) Novel compounds, pharmaceutical compositions containing same, and methods of use for same

(76) Inventors: Francis P. Kuhadja, Baltimore, MD (US); Susan M. Medghalchi, Baltimore, MD (US); Jagan N. Thupari, Baltimore, MD (US); Craig A. Townsend, Baltimore, MD (US); Jill M. McFadden, Baltimore, MD (US)

Correspondence Address: COVINGTON & BURLING, LLP;ATTN: PATENT DOCKETING, 1201 PENNSYLVANIA AVENUE, N.W., WASHINGTON, DC, 20004-2401, US

(21) Appl. No.: 10/519,804

(22) PCT Filed: Jul. 1, 2003
(86) PCT No.: PCT/US03/20960

§ 371 Date: May 19, 2006

Related U.S. Application Data
(60) Provisional application No. 60/392,809, filed on Jul. 1, 2002.

(51) Int. Cl. A61K 31/365; C07D 305/12

(52) U.S. Cl. 514/471; 514/473; 549/321

(57) ABSTRACT

Pharmaceutical composition comprising a pharmaceutical diluent and a compound of formula IX: R²⁹═H, or C₁-C₂₀ alkyl, cycloalkyl, alkenyl, aryl, arylalkyl, or alkylaryl, ═CHR³¹, —C(O)OR³¹, —C(O)R³¹, —CH₂C(O)OR³¹, CH₂C(O)NHR³¹, where R³¹ is H or C₁-C₁₀ alkyl, cycloalkyl, or alkenyl; R³⁰═C₁-C₂₀ alkyl, cycloalkyl, alkenyl, aryl, arylalkyl, or alkylaryl; X⁵═—OR³², or NHR³², Where R³² is H, C₁-C₂₀ alkyl, cycloalkyl, alkenyl, aryl, arylalkyl, or alkylaryl, the R³² group optionally containing a carbonyl group, a carboxyl group, a carboxyamide group, an alcohol group, or an ether group, the R³² group further optionally containing one or more halogen atoms; with the proviso that when R²⁹ is ═CH₂, then X⁵ is not OH. Also disclosed are compounds within the scope of the formula IX, as well as uses of the pharmaceutical compositions for weight loss, anti-microbial and anti-cancer applications, inhibition of fatty acid synthase and neuropeptide-Y, and the stimulation of the activity of carnitine palmitoyl transferase-1.

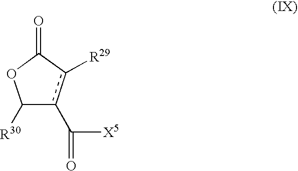

### BACKGROUND OF THE INVENTION

Fatty Acid Synthase

Fatty acids have three primary roles in the physiology of cells. First, they are the building bocks of biological membranes. Second, fatty acid derivatives serve as hormones and intracellular messengers. Third, and of particular importance to the present invention, fatty acids are fuel molecules that can be stored in adipose tissue as triacylglycerols, which are also known as neutral fats.

There are four primary enzymes involved in the fatty acid synthetic pathway, fatty acid synthase (FAS), acetyl CoA carboxylase (ACC), malic enzyme, and citric lyase. The principal enzyme, FAS, catalyzes the NADPH-dependent condensation of the precursors malonyl-CoA and acetyl-CoA to produce fatty acids. NADPH is a reducing agent that generally serves as the essential electron donor at two points in the reaction cycle of FAS. The other three enzymes (i.e., ACC, malic enzyme, and citric lyase) produce the necessary precursors. Other enzymes, for example the enzymes that produce NADPH, are also involved in fatty acid synthesis.

FAS has an Enzyme Commission (E.C.) No. 2.3.1.85 and is also known as fatty acid synthetase, fatty acid ligase, as well as its systematic name acyl-CoA:malonyl-CoA C-acyltransferase (decarboxylating, oxoacyl- and enoyl-reducing and thioester-hydrolysing). There are seven distinct enzymes—or catalytic domains—involved in the FAS catalyzed synthesis of fatty acids: acetyl transacylase, malonyl transacylase, beta-ketoacyl synthetase (condensing enzyme), beta-ketoacyl reductase, beta-hydroxyacyl dehydrase, enoyl reductase, and thioesterase. (Wakil, S. J., Biochemistry, 28: 4523-4530, 1989). All seven of these enzymes together form FAS.

Although the FAS catalyzed synthesis of fatty acids is similar in lower organisms, such as, for example, bacteria, and in higher organisms, such as, for example, mycobacteria, yeast and humans, there are some important differences. In bacteria, the seven enzymatic reactions are carried out by seven separate polypeptides that are non-associated. This is classified as Type II FAS. In contrast, the enzymatic reactions in mycobacteria, yeast and humans are carried out by multifunctional polypeptides. For example, yeast have a complex composed of two separate polypeptides whereas in mycobacterium and humans, all seven reactions are carried out by a single polypeptide. These are classified as Type I FAS.

FAS Inhibitors

Various compounds have been shown to inhibit fatty acid synthase (FAS). FAS inhibitors can be identified by the ability of a compound to inhibit the enzymatic activity of purified FAS. FAS activity can be assayed by measuring the incorporation of radiolabeled precursor (i.e., acetyl-CoA or malonyl-CoA) into fatty acids or by spectrophotometrically measuring the oxidation of NADPH. (Dils, et al., Methods Enzymol., 35:74-83).

Table 1, set forth below, lists several FAS inhibitors.

TABLE 1
Representative Inhibitors Of The Enzymes
Of The Fatty Acid Synthesis Pathway

| | |
|---|---|
| Inhibitors of Fatty Acid Synthase | |
| 1,3-dibromopropanone | cerulenin |
| Ellman's reagent (5,5′-dithiobis(2- | phenyocerulenin |
| nitrobenzoic acid), DTNB) | melarsoprol |
| 4-(4′-chlorobenzyloxy) benzyl | iodoacetate |
| nicotinate (KCD-232) | phenylarshineoxide |
| 4-(4′-chlorobenzyloxy) benzoic | pentostam |
| acid (MII) | melittin |
| 2(5(4-chlorophenyl)pentyl)oxirane- | thiolactomycin |
| 2-carboxylate (POCA) and its CoA | |
| derivative | |
| ethoxyformic anhydride | |
| Inhibitors for citrate lyase | Inhibitors for malic enzyme |
| (−) hydroxycitrate | periodate-oxidized 3- |
| (R,S)-S-(3,4-dicarboxy-3-hydroxy- | aminopyridine adenine |
| 3-methyl-butyl)-CoA | dinucleotide phosphate |
| S-carboxymethyl-CoA | 5,5′-dithiobis(2-nitrobenzoic |
| | acid) |
| | p-hydroxymercuribenzoate |
| | N-ethylmaleimide |
| | oxalyl thiol esters such as |
| | S-oxalylglutathione |
| | gossypol |
| | phenylglyoxal |
| | 2,3-butanedione |
| | bromopyruvate |
| | pregnenolone |
| Inhibitors for acetyl CoA | |
| carboxylase | |
| sethoxydim | 9-decenyl-1-pentenedioic acid |
| haloxyfop and its CoA ester | decanyl-2-pentenedioic acid |
| diclofop and its CoA ester | decanyl-1-pentenedioic acid |
| clethodim | (S)-ibuprofenyl-CoA |
| alloxydim | (R)-ibuprofenyl-CoA |
| trifop | fluazifop and its CoA ester |
| clofibric acid | clofop |
| 2,4-D mecoprop | 5-(tetradecycloxy)-2-furoic |
| dalapon | acid |
| 2-alkyl glutarate | beta, beta′- |
| 2-tetradecanylglutarate (TDG) | tetramethylhexadecanedioic |
| 2-octylglutaric acid | acid |
| N6,02-dibutyryl adenosine cyclic | tralkoxydim |
| 3′,5′-monophosphate | free or monothioester of |
| N2,02-dibutyryl guanosine cyclic | beta, beta prime-methyl- |
| 3′,5′-monophosphate | substituted hexadecanedioic |
| CoA derivative of 5-(tetradecyloxy)- | acid (MEDICA 16) |
| 2-furoic acid (TOFA) | alpha-cyanco-4- |
| 2,3,7,8-tetrachlorodibenzo-p-dioxin | hydroxycinnamate |
| | S-(4-bromo-2,3-dioxobutyl)- |
| | CoA |
| | p-hydroxymercuribenzoate |
| | (PHMB) |
| | N6,02-dibutyryl adenosine |
| | cyclic 3′,5′-monophosphate |

Of the four enzymes in the fatty acid synthetic pathway, FAS is the preferred target for inhibition because it acts only within the pathway to fatty acids, while the other three enzymes are implicated in other cellular functions. Therefore, inhibition of one of the other three enzymes is more likely to affect normal cells. Of the seven enzymatic steps carried out by FAS, the step catalyzed by the condensing enzyme (i.e., beta-ketoacyl synthetase) and the enoyl reductase have been the most common candidates for inhibitors that reduce or stop fatty acid synthesis. The condensing enzyme of the FAS complex is well characterized in terms of structure and function. The active site of the condensing enzyme contains a critical cysteine thiol, which is the target of antilipidemic reagents, such as, for example, the inhibitor cerulenin.

Preferred inhibitors of the condensing enzyme include a wide range of chemical compounds, including alkylating agents, oxidants, and reagents capable of undergoing disulphide exchange. The binding pocket of the enzyme prefers long chain, E, E, dienes.

In principal, a reagent containing the sidechain diene and a group which exhibits reactivity with thiolate anions could be a good inhibitor of the condensing enzyme. Cerulenin [(2S,3R)-2,3-epoxy-4-oxo-7,10 dodecadienoyl amide] is an example: 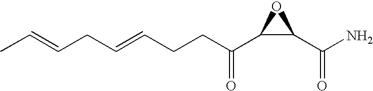
Cerulenin covalently binds to the critical cysteine thiol group in the active site of the condensing enzyme of fatty acid synthase, inactivating this key enzymatic step (Funabashi, et al., J. Biochem., 105:751-755, 1989). While cerulenin has been noted to possess other activities, these either occur in microorganisms which may not be relevant models of human cells (e.g., inhibition of cholesterol synthesis in fungi, Omura (1976), Bacteriol. Rev., 40:681-697; or diminished RNA synthesis in viruses, Perez, et al. (1991), FEBS, 280: 129-133), occur at a substantially higher drug concentrations (inhibition of viral HIV protease at 5 mg/ml, Moelling, et al. (1990), FEBS, 261:373-377) or may be the direct result of the inhibition of endogenous fatty acid synthesis (inhibition of antigen processing in B lymphocytes and macrophages, Falo, et al. (1987), J. Immunol., 139:3918-3923). Some data suggest that cerulenin does not specifically inhibit myristoylation of proteins (Simon, et al., J. Biol. Chem., 267:3922-3931, 1992).

Several more FAS inhibitors are disclosed in U.S. patent application Ser. No. 08/096,908 and its CIP filed Jan. 24, 1994, the disclosures of which are hereby incorporated by reference. Included are inhibitors of fatty acid synthase, citrate lyase, CoA carboxylase, and malic enzyme.

Tomoda and colleagues (Tomoda et. al., Biochim. Biophys. Act 921:595-598 1987; Omura el. al., J. Antibiotics 39:1211-1218 1986) describe Triacsin C (sometimes termed WS-1228A), a naturally occurring acyl-CoA synthetase inhibitor, which is a product of Streptomyces sp. SK-1894. The chemical structure of Triacsin C is 1-hydroxy-3-(E, E, E-2′,4′,7′-undecatrienylidine) triazene. Triacsin C causes 50% inhibition of rat liver acyl-CoA synthetase at 8.7 μM; a related compound, Triacsin A, inhibits acyl CoA-synthetase by a mechanism which is competitive with long-chain fatty acids. Inhibition of acyl-CoA synthetase is toxic to animal cells. Tomoda et al. (Tomoda el. al., J. Biol. Chem. 266:4214-4219, 1991) teaches that Triacsin C causes growth inhibition in Raji cells at 1.0 μM, and have also been shown to inhibit growth of Vero and Hela cells. Tomoda el. al. further teaches that acyl-CoA synthetase is essential in animal cells and that inhibition of the enzyme has lethal effects.

A family of compounds (gamma-substituted-alpha-methylene-beta-carboxy-gamma-butyrolactones) has been shown in U.S. Pat. No. 5,981,575 (the disclosure of which is hereby incorporated by reference) to inhibit fatty acid synthesis, inhibit growth of tumor cells, and induce weight loss. The compounds disclosed in the '575 Patent have several advantages over the natural product cerulenin for therapeutic applications: [1] they do not contain the highly reactive epoxide group of cerulenin, [2] they are stable and soluble in aqueous solution, [3] they can be produced by a two-step synthetic reaction and thus easily produced in large quantities, and [4] they are easily tritiated to high specific activity for biochemical and pharmacological analyses. The synthesis of this family of compounds, which are fatty acid synthase inhibitors, is described in the '575 Patent, as is their use as a means to treat tumor cells expressing FAS, and their use as a means to reduce body weight. The '575 Patent also discloses the use of any fatty acid synthase inhibitors to systematically reduce adipocyte mass (adipocyte cell number or size) as a means to reduce body weight.

The primary sites for fatty acid synthesis in mice and humans are the liver (see Roncari, Can. J. Biochem., 52:221-230, 1974; Triscari et al., 1985, Metabolism, 34:580-7; Barakat et al., 1991, Metabolism, 40:280-5), lactating mammary glands (see Thompson, et al., Pediatr. Res., 19:139-143, 1985) and adipose tissue (Goldrick et al., 1974, Clin. Sci. Mol. Med., 46:469-79).

Inhibitors of Fatty Acid Synthesis as Antimicrobial Agents

Cerulenin was originally isolated as a potential antifungal antibiotic from the culture broth of Cephalosporium caerulens. Structurally cerulenin has been characterized as (2R,3S)-epoxy-4-oxo-7,10-trans,trans-dodecanoic acid amide. Its mechanism of action has been shown to be inhibition, through irreversible binding, of beta-ketoacyl-ACP synthase, the condensing enzyme required for the biosynthesis of fatty acids. Cerulenin has been categorized as an antifungal, primarily against Candida and Saccharomyces sp. In addition, some in vitro activity has been shown against some bacteria, actinomycetes, and mycobacteria, although no activity was found against Mycobacterium tuberculosis. The activity of fatty acid synthesis inhibitors and cerulenin in particular has not been evaluated against protozoa such as Toxoplasma gondii or other infectious eucaryotic pathogens such as Pneumocystis carinii, Giardia lamblia, Plasmodium sp., Trichomonas vaginalis, Cryptosporidium, Trypanosoma, Leishmania, and Schistosoma.

Infectious diseases which are particularly susceptible to treatment are diseases which cause lesions in externally accessible surfaces of the infected animal. Externally accessible surfaces include all surfaces that may be reached by non-invasive means (without cutting or puncturing the skin), including the skin surface itself, mucus membranes, such as those covering nasal, oral, gastrointestinal, or urogenital surfaces, and pulmonary surfaces, such as the alveolar sacs. Susceptible diseases include: (1) cutaneous mycoses or tineas, especially if caused by Microsporum, Trichophyton, Epidermophyton, or Mucocutaneous candidiasis; (2) mucotic keratitis, especially if caused by Aspergillus, Fusarium or Candida; (3) amoebic keratitis, especially if caused by Acanthamoeba; (4) gastrointestinal disease, especially if caused by Giardia lamblia, Entamoeba, Cryptosporidium, Microsporidium, or Candida (most commonly in immunocompromised animals); (5) urogenital infection, especially if caused by Candida albicans or Trichomonas vaginalis; and (6) pulmonary disease, especially if caused by Mycobacterium tuberculosis, Aspergillus, or Pneumocystis carinii. Infectious organisms that are susceptible to treatment with fatty acid synthesis inhibitors include Mycobacterium tuberculosis, especially multiply-drug resistant strains, and protozoa such as Toxoplasma.

Any compound that inhibits fatty acid synthesis may be used to inhibit microbial cell growth. However, compounds administered to a patient must not be equally toxic to both patient and the target microbial cells. Accordingly, it is beneficial to select inhibitors that only, or predominantly, affect target microbial cells.

Eukaryotic microbial cells which are dependent on their own endogenously synthesized fatty acid will express Type I FAS. This is shown both by the fact that FAS inhibitors are growth inhibitory and by the fact that exogenously added fatty acids can protect normal patient cells but not these microbial cells from FAS inhibitors. Therefore, agents which prevent synthesis of fatty acids by the cell may be used to treat infections. In eukaryotes, fatty acids are synthesized by Type I FAS using the substrates acetyl CoA, malonyl CoA and NADPH. Thus, other enzymes which can feed substrates into this pathway may also effect the rate of fatty acid synthesis and thus be important in microbes that depend on endogenously synthesized fatty acid. Inhibition of the expression or activity of any of these enzymes will effect growth of the microbial cells that are dependent upon endogenously synthesized fatty acid.

The product of Type I FAS differs in various organisms. For example, in the fungus S. cerevisiae the products are predominately palmitate and sterate sterified to coenzyme-A. In Mycobacterium smegmatis, the products are saturated fatty acid CoA esters ranging in length from 16 to 24 carbons. These lipids are often further processed to fulfill the cells need for various lipid components.

Inhibition of key steps in down-stream processing or utilization of fatty acids may be expected to inhibit cell function, whether the cell depends on endogenous fatty acid or utilizes fatty acid supplied from outside the cell, and so inhibitors of these down-stream steps may not be sufficiently selective for microbial cells that depend on endogenous fatty acid. However, it has been discovered that administration of Type I fatty acid synthesis inhibitor to such microbes makes them more sensitive to inhibition by inhibitors of down-stream fatty acid processing and/or utilization. Because of this synergy, administration of a fatty acid synthesis inhibitor in combination with one or more inhibitors of down-stream steps in lipid biosynthesis and/or utilization will selectively affect microbial cells that depend on endogenously synthesized fatty acid. Preferred combinations include an inhibitor of FAS and acetyl CoA carboxylase, or FAS and an inhibitor of MAS.

When it has been determined that a mammal is infected with cells of an organism which expresses Type I FAS, or if FAS has been found in a biological fluid from a patient, the mammal or patient maybe treated by administering a fatty acid synthesis inhibitor (U.S. Pat. No. 5,614,551).

The inhibition of neuropeptide-Y to depress appetite and stimulate weight loss is described in International Patent Application No. PCT/US01/05316 the disclosure of which is hereby incorporated by reference. That application, however, does not describe or disclose any of the compounds disclosed in the present application

The stimulation of carnitine palmitoyl transferase-1 (CPT-1) to stimulate weight loss is described in U.S. Patent Application Ser. No. 60/354,480, the disclosure of which is hereby incorporated by reference. That application does not describe or disclose any of the compounds disclosed herein, either.

The use of FAS inhibitors to inhibit the growth of cancer cells is described in U.S. Pat. No. 5,759,837, the disclosure of which is hereby incorporated by reference. That application does not describe or disclose any of the compounds disclosed herein.

### SUMMARY OF THE INVENTION

New classes of compounds have been discovered which have a variety of therapeutically valuable properties, eg. FAS-inhibition, NPY-inhibition, CPT-1 stimulation, ability to induce weight loss, and anti-cancer and anti-microbial properties.

It is a further object of this invention to provide a method of inducing weight loss in animals and humans by administering a pharmaceutical composition comprising a pharmaceutical diluent and a compound of formula I, II, III, IV, V, VI, VII, VIII, or IX, which are described in detail below.

It is a further object of the invention to provide a method of stimulating the activity of CPT-1 by administering to humans or animals a pharmaceutical composition comprising a pharmaceutical diluent and a compound of formula I, II, III, IV, V, VI, VII, VIII, or IX.

It is a further object of the invention to provide a method of inhibiting the synthesis of neuropeptide Y in humans or animals by administering a pharmaceutical composition comprising a pharmaceutical diluent and a compound of formula I, II, III, IV, V, VI, VII, VIII, or IX.

It is a further object of the invention to provide a method of inhibiting fatty acid synthase activity in humans or animals by administering a pharmaceutical composition comprising a pharmaceutical diluent and a compound of formula I, II, III, IV, V, VI, VII, VIII, or IX.

It is a further object of this invention to provide a method of treating cancer in animals and humans by administering a pharmaceutical composition comprising a pharmaceutical diluent and a compound of formula I, II, III, IV, V, VI, VII, VIII, or IX.

It is still a further object of this invention to provide a method of preventing the growth of cancer cells in animals and humans by administering a pharmaceutical composition comprising a pharmaceutical diluent and a compound of formula I, II, III, IV, V, VI, VII, VIII, or IX.

It is a further object of this invention to provide a method of inhibiting growth of invasive microbial cells by administering a pharmaceutical composition comprising a pharmaceutical diluent and a compound of compound of formula I, II, III, IV, V, VI, VII, VIII, or IX.

### BRIEF DESCRIPTION OF THE DRAWINGS

FIG. 1 shows a synthetic scheme to make certain compounds according to the invention.

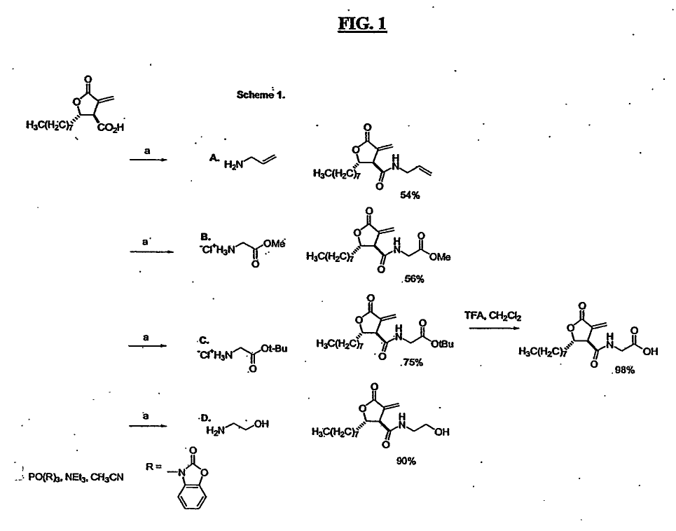

FIG. 2 shows a synthetic scheme to make certain compounds according to the invention

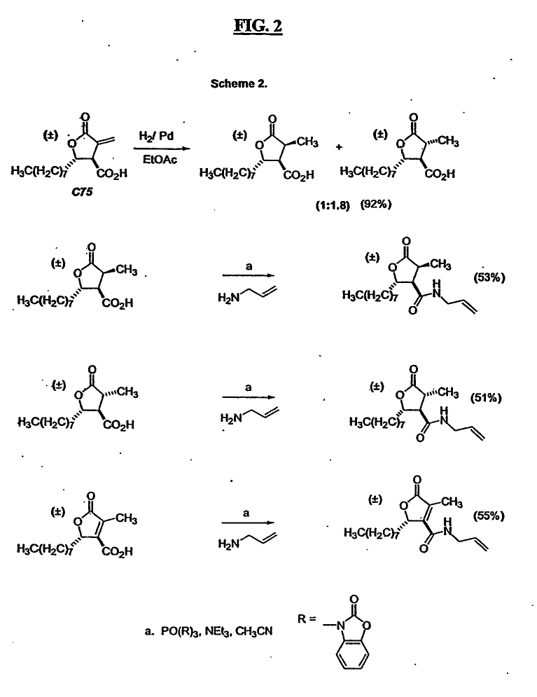

FIG. 3 shows the results of in vivo testing of the anti-tumor properties of certain compounds according to the invention.

FIG. 4 shows the results of in vivo testing of the anti-tumor properties of a different compound according to the invention.

FIG. 5 shows the results of in vivo testing for weight loss of certain compounds according to the invention.

### DETAILED DESCRIPTION OF THE INVENTION

The compounds of the invention can be prepared by conventional means. The synthesis of a number of the compounds is described in the examples. The compounds may be useful for the treatment of obesity, cancer, or microbially-based infections.

One embodiment of the invention is compounds of formula I: 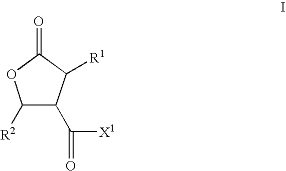
wherein
R¹═H, or C₁-C₂₀ alkyl, cycloalkyl, alkenyl, aryl, arylalkyl, or alkylaryl, ═CHR³, —C(O)OR³, —C(O)R³, —CH₂C(O)OR³, —CH₂C(O)NHR³, where R³ is H or C₁-C₁₀ alkyl, cycloalkyl, or alkenyl;
R²═C₁-C₂₀ alkyl, cycloalkyl, alkenyl, aryl, arylalkyl, or alkylaryl;
X¹═NHR⁴, where R⁴ is H, C₁-C₁₀ alkyl, cycloalkyl, alkenyl, aryl, arylalkyl, or alkylaryl, the R⁴ group optionally containing a carbonyl group, a carboxyl group, a carboxyamide group, an alcohol group, or an ether group, the R⁴ group further optionally containing one or more halogen atoms.

In a preferred embodiment, R¹ is C₁-C₁₀ alkyl, cycloalkyl, alkenyl, aryl, arylalkyl, or alkylaryl; or ═CH₂. In a more preferred embodiment, R¹ is —CH₃ or ═CH₂.

In another preferred embodiment, R⁴ is —CH₂C(O)OR⁵ or —CH₂C(O)NHR⁵, where R⁵ is C₁-C₁₀ alkyl, cycloalkyl, alkenyl, aryl, arylalkyl, or alkylaryl.

Another embodiment of the invention is compounds formula II 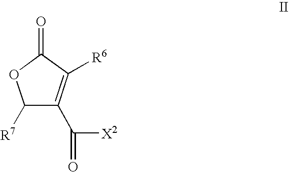
wherein
R⁶═H, or C₁-C₂₀ alkyl cycloalkyl, alkenyl, aryl, arylalkyl, or alkylaryl, —C(O)OR⁸, —C(O)R⁸, —CH₂C(O)OR⁸, —CH₂C(O)NHR⁸, where R⁸ is H or C₁-C₁₀ alkyl, cycloalkyl, or alkenyl;
R⁷═C₁-C₂₀ alkyl, cycloalkyl, alkenyl, aryl, arylalkyl, or alkylaryl;
X²═NHR⁹, where R⁹ is H, C₁-C₂₀ alkyl, cycloalkyl, alkenyl, aryl, arylalkyl, or alkylaryl, the R⁹ group optionally containing a carbonyl group, a carboxyl group, a carboxyamide group, an alcohol group, or an ether group, the R⁹ group further optionally containing one or more halogen atoms;
with the proviso that when R⁶ is —CH₃, and R⁷ is n-C₁₃H₂₇, X² is not —NHC₂H₅.

In a preferred embodiment, R⁶ is C₁-C₁₀ alkyl, cycloalkyl, alkenyl, aryl, arylalkyl, or alkylaryl. In a more preferred embodiment, R⁶ is —CH₃.

In another preferred embodiment, R⁹ is —CH₂C(O)OR¹⁰ or —CH₂C(O)NHR¹⁰, where R¹⁰ is C₁-C₁₀ alkyl, cycloalkyl, alkenyl, aryl, arylalkyl, or alkylaryl.

Another embodiment of the invention is compounds of formula III: 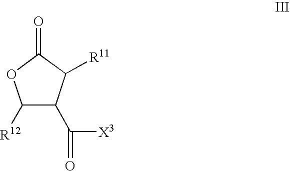
wherein
R¹¹═H, or C₁-C₂₀ alkyl, cycloalkyl, alkenyl, aryl, arylalkyl, or alkylaryl, ═CHR¹³, —C(O)OR¹³, —C(O)R¹³, —CH₂C(O)OR¹³, —CH₂C(O)NHR¹³, where R¹³ is H or C₁-C₁₀ alkyl, cycloalkyl, or alkenyl;
R¹²═C₁-C₂₀ alkyl cycloalkyl, alkenyl, aryl, arylalkyl, or alkylaryl;
X³═OR¹⁴, where R¹⁴ is C₁-C₂₀ alkyl, cycloalkyl, alkenyl, aryl, arylalkyl, or alkylaryl, the R¹⁴ group optionally containing a carbonyl group, a carboxyl group, a carboxyamide group, an alcohol group, or an ether group, the R¹⁴ group further optionally containing one or more halogen atoms.

In a preferred embodiment, R¹¹ is C₁-C₁₀ alkyl, cycloalkyl, alkenyl, aryl, arylalkyl, or alkylaryl; or ═CH₂. In a more preferred embodiment, R¹¹ is —CH₃ or ═CH₂.

In another preferred embodiment, R¹⁴ is —CH₂C(O)OR¹⁵ or —CH₂C(O)NHR¹⁵, where R¹⁵ is C₁-C₁₀ alkyl, cycloalkyl, alkenyl, aryl, arylalkyl, or alkylaryl.

Another embodiment of the invention is compounds of formula IV: 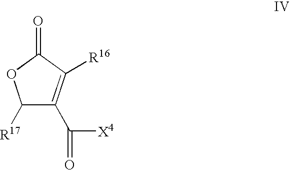
wherein
R¹⁶═H, or C₁-C₂₀ alkyl, cycloalkyl, alkenyl, aryl, arylalkyl, or alkylaryl, —C(O)OR¹⁸, —C(O)R¹⁸, —CH₂C(O)OR¹⁸, —CH₂C(O)NHR¹⁸, where R¹⁸ is H or C₁-C₁₀ alkyl, cycloalkyl, or alkenyl;
R¹⁷═C₁-C₂₀ alkyl, cycloalkyl, alkenyl, aryl, arylalkyl, or alkylaryl;
X⁴═OR¹⁹, where R¹⁹ is C₁-C₂₀ alkyl, cycloalkyl, alkenyl, aryl, arylalkyl, or alkylaryl, the R¹⁹ group optionally containing a carbonyl group, a carboxyl group, a carboxyamide group, an alcohol group, or an ether group, the R¹⁹ group further optionally containing one or more halogen atoms,
with the proviso that when R¹⁶ is —CH₃ and R¹⁹ is —CH₃, then R¹⁷ is not substituted or unsubstituted phenyl, -nC₃H₇, -nC₅H₁₁, or -nC₁₃H₂₇,
and with the further proviso that when R¹⁶ is H and R¹⁹ is —CH₃, then R¹⁷ is not substituted or unsubstuted phenyl or —CH₃, and when R¹⁶ is H and R¹⁹ is —CH₂CH₃, then R¹⁷ is not -iC₃H₇, or substituted or unsubstituted phenyl.

In a preferred embodiment, R¹⁶ is C₁-C₁₀ alkyl, cycloalkyl, alkenyl, aryl, arylalkyl, or alkylaryl. In a more preferred embodiment, R¹⁶ is —CH₃.

In another preferred embodiment, R¹⁹ is —CH₂C(O)OR²⁰ or —CH₂C(O)NHR²⁰, where R²⁰ is C₁-C₁₀ alkyl, cycloalkyl, alkenyl, aryl, arylalkyl, or alkylaryl.

Another embodiment of the invention is compounds of formula V: 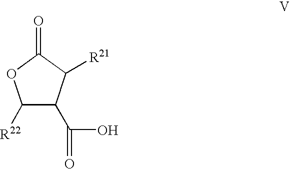
wherein
R²¹═C₂-C₂₀ alkyl, cycloalkyl, alkenyl, aryl, arylalkyl, or alkylaryl, ═CHR²³, —C(O)OR²³, —C(O)R²³, —CH₂C(O)OR²³, —CH₂C(O)NHR²³, where R²³ is H or C₁-C₁₀ alkyl, cycloalkyl, or alkenyl, except when R²¹ is ═CHR²³, R²³ is not H;
R²²═C₁-C₂₀ alkyl, cycloalkyl, alkenyl, aryl, arylalkyl, or alkylaryl;
with the proviso that when R²¹ is —COOH, then R²² is not —CH₃, -nC₅H₁₁, or C₁₃H₂₇, and with the further proviso that when R²¹ is —CH₂COOH, then R²² is not —CH₃, —CH₂CH₃, or -iC₅H₁₁, and the further proviso that when R²¹ is ═CHCH₃, then R²² is not n-C₅H₁₁.

In a preferred embodiment, R²¹ is C₂-C₁₀ alkyl, cycloalkyl, alkenyl, aryl, arylalkyl, or alkylaryl.

Another embodiment of the invention is compounds of formula VI: 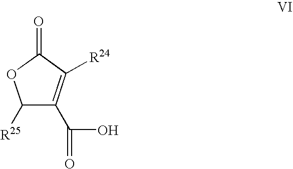
wherein
R²⁴═C₂-C₂₀ alkyl, cycloalkyl, alkenyl, aryl, arylalkyl, or alkylaryl, —C(O)OR²⁶, —C(O)R²⁶, —CH₂C(O)OR²⁶, —CH₂C(O)NHR²⁶, where R²⁶ is H or C₁-C₁₀ alkyl, cycloalkyl, or alkenyl;
R²⁵═C₁-C₂₀ alkyl, cycloalkyl, alkenyl, aryl, arylalkyl, or alkylaryl;
with the proviso that when R²⁴ is —COOH, then R²⁵ is not —CH₃, -nC₅H₁₁, or C₁₃H₂₇, and with the further proviso that when R²⁴ is —CH₂COOH, then R²⁵ is not —CH₃, —CH₂CH₃, or -iC₅H₁₁.

In a preferred embodiment, R²¹ is C₂-C₁₀ alkyl, cycloalkyl, alkenyl, aryl, arylalkyl, or alkylaryl.

Another embodiment of the invention is compounds of formula VII: 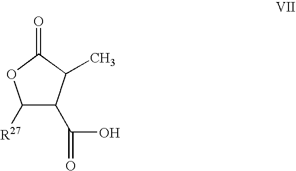
wherein
R²⁷═C₃-C₄ alkyl, C₆-C₁₀ alkyl, C₁₂ alkyl, C₁₄ alkyl, C₁₆-C₂₀ alkyl.

Another embodiment of the invention is compounds of formula VIII: 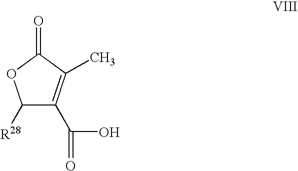
wherein R²⁸ is C₁-C₂₀ alkyl, cycloalkyl, alkenyl, aryl, arylalkyl, or alkylaryl, with the proviso that R²⁸ is not CH₃, -nC₃H₇, -nC₁₁H₂₃, or -nC₁₃H₂₇.

Another embodiment of the invention is pharmaceutical compositions comprising a pharmaceutical diluent or carrier and a compound of formula I, II, III, IV, V, VI, VII, VIII, or IX: 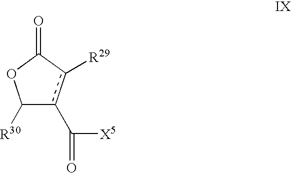
R²⁹═H, or C₁-C₂₀ alkyl, cycloalkyl, alkenyl, aryl, arylalkyl, or alkylaryl, ═CHR³¹, —C(O)OR³¹, —C(O)R³¹, —CH₂C(O)OR³¹, —CH₂C(O)NHR³¹, where R³¹ is H or C₁-C₁₀ alkyl, cycloalkyl, or alkenyl;
R³⁰═C₁-C₂₀ alkyl, cycloalkyl, alkenyl, aryl arylalkyl, or alkylaryl;
X⁵═—OR³², or —NHR³², where R³² is H, C₁-C₂₀ alkyl, cycloalkyl, alkenyl, aryl, arylalkyl or alkylaryl, the R³² group optionally containing a carbonyl group, a carboxyl group, a carboxyamide group, an alcohol group, or an ether group, the R³² group further optionally containing one or more halogen atoms;
with the proviso that when R²⁹ is ═CH₂, then X⁵ is not —OH.

In a preferred embodiment, R²⁹ is C₁-C₁₀ alkyl, cycloalkyl alkenyl, aryl, arylalkyl, or alkylaryl, or ═CH₂. In a more preferred embodiment, R²⁹ is —CH₃ or ═CH₂.

In another preferred embodiment, R³² is —CH₂C(O)OR³³ or —CH₂C(O)NHR³³, where R³³ is C₁-C₁₀ alkyl, cycloalkyl, alkenyl, aryl, arylalkyl, or alkylaryl.

The compositions of the present invention can be presented for administration to humans and other animals in unit dosage forms, such as tablets, capsules, pills, powders, granules, sterile parenteral solutions or suspensions, oral solutions or suspensions, oil in water and water in oil emulsions containing suitable quantities of the compound, suppositories and in fluid suspensions or solutions. As used in this specification, the terms “pharmaceutical diluent” and “pharmaceutical carrier,” have the same meaning. For oral administration, either solid or fluid unit dosage forms can be prepared. For preparing solid compositions such as tablets, the compound can be mixed with conventional ingredients such as talc, magnesium stearate, dicalcium phosphate, magnesium aluminum silicate, calcium sulfate, starch, lactose, acacia, methylcellulose and functionally similar materials as pharmaceutical diluents or carriers. Capsules are prepared by mixing the compound with an inert pharmaceutical diluent and filling the mixture into a hard gelatin capsule of appropriate size. Soft gelatin capsules are prepared by machine encapsulation of a slurry of the compound with an acceptable vegetable oil, light liquid petrolatum or other inert oil.

Fluid unit dosage forms or oral administration such as syrups, elixirs, and suspensions can be prepared. The forms can be dissolved in an aqueous vehicle together with sugar, aromatic flavoring agents and preservatives to form a syrup. Suspensions can be prepared with an aqueous vehicle with the aid of a suspending agent such as acacia, tragacanth, methylcellulose and the like.

For parenteral administration fluid unit dosage forms can be prepared utilizing the compound and a sterile vehicle. In preparing solutions the compound can be dissolved in water for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The lyophilized powder can then be scaled in the vial and reconstituted prior to use.

The clinical therapeutic indications envisioned for the compounds of the invention include: (1) infections due to invasive micro-organisms such as staphylococci and enterococci; (2) cancers arising in many tissues whose cells over-express fatty acid synthase, and (3) obesity due to the ingestion of excess calories. Dose and duration of therapy will depend on a variety of factors, including (1) the patient's age, body weight, and organ function (e.g., liver and kidney function); (2) the nature and extent of the disease process to be treated, as well as any existing significant co-morbidity and concomitant medications being taken, and (3) drug-related parameters such as the route of administration, the frequency and duration of dosing necessary to effect a cure, and the therapeutic index of the drug. In general, does will be chosen to achieve serum levels of 1 ng/ml to 100 ng/ml with the goal of attaining effective concentrations at the target site of approximately 1 μg/ml to 10 μg/ml.

### EXAMPLES

The invention will be illustrated, but not limited, by the following examples:

A series of compounds according to the invention were synthesized as described below. Biological activity of certain compounds were profiled as follows: Compounds were tested for: (1) inhibition of purified human FAS, (2) inhibition of fatty acid synthesis activity in whole cells, (3) cytotoxicity against cultured MCF-7 human breast cancer cells, known to possess high levels of FAS and fatty acid synthesis activity, using the crystal violet and XTT assays, and (4) antimicrobial activity. Select compounds with low levels of cytotoxicity were then tested for weight loss in Balb/C mice. In addition, a representative compound from the group which exhibited significant weight loss and low levels of cytotoxicity was tested for its effect on fatty acid oxidation, and carnitine palmitoyltransferase-1 (CPT-1) activity, as well as hypothalamic NPY expression by Northern analysis in Balb/C mice. Certain compounds were also tested for activity against gram positive and/or negative bacteria. Certain compounds were also tested in vivo for anti-tumor activity.
Preparation of the Compounds

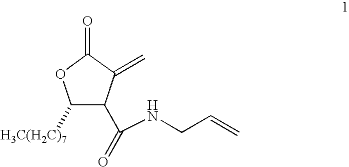

### (±)-α-Methylene-γ-butyrolactone-5-octyl-4-allyl amide

(1) To a solution of (±)-α-Methylene-γ-butyrolactone-5-octyl-4-carboxylic acid (C75), (40 mg, 0.16 mmol) in CH₃CN (0.9 mL) was added tris (2-oxo-3-oxazolinyl)phosphine oxide¹ (91.7 mg, 0.2 mmol), allylamine (12 μl, 0.2 mmol) and NEt₃ (0.04 mL, 0.3 mmol) and the solution was allowed to stir for 30 min at it. The mixture was poured into a solution of NH₄Cl₍ₛₐₜ₎/1 N HCl (10 mL, 3:1) and extracted with Et₂O (3×15 mL). The combined organics were dried (MgSO₄), filtered, evaporated and chromatographed (35% EtOAc/Hexanes) to give pure 1 (26.2 mg, 54%); mp. 66-68° C. ¹H NMR (300 MHz, CDCl₃) δ 0.84 (t, J=6 Hz, 3 H), 1.23 (m, 11 H), 1.34-1.47 (m, 1 H), 1.60-1.71 (m, 2 H), 3.43-3.46 (m, 1 H), 3.87 (dt, J=1.4, 5.7 Hz, 2 H), 4.74 (dt, J=5, 7 Hz, 1 H), 5.12 (d, J=10.6 Hz, 1 H), 5.16 (d, J=17.3 Hz, 1 H), 5.72-5.85 (m, 1 H), 5.76 (d, J=2.6 Hz, 1 H), 6.34 (d, J=2.6 Hz, 1 H), 6.50 (bs, 1 H). ¹³C NMR (75 MHz, CDCl₃) δ 14.0, 22.6, 24.9, 29.1, 29.2, 29.4, 31.8, 35.9, 42.3, 52.2, 80.5, 117.0, 124.3, 133.5, 135.4, 168.6, 168.6. IR (NaCl) 2922, 1771, 1756, 1642 1557 cm⁻¹. Anal. Calcd for C₁₇H₂₇NO₃: C, 69.5; H, 9.28; Found: C, 69.5; H, 9.09.

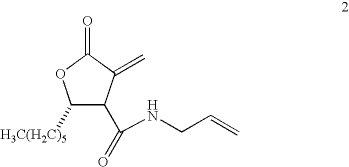

### (±)-α-Methylene-γ-butyrolactone-5-hexyl-4-allyl amide (2)

From (±)-α-Methylene-γ-butyrolactone-5-hexyl-4-carboxylic acid. (60 mg, 0.27 mmol) and allyl amine (33 μL, 0.29 mmol) following the above procedure was obtained 2 (51.8 mg, 74%) after flash chromatography (30-40% EtOAc/Hexanes). ¹H NMR (300 M , CDCl₃) δ 0.86 (t, J=6 Hz, 3H), 1.26-1.52 (m, 8 H), 1.63-1.77 (m, 2 H), 3.40-3.43 (m, 1 H), 3.91 (app tt, J=5.76, 1.44 Hz, 2 H),4.72-4.78 (m, 1 H), 5.14-5.20 (m, 2 H), 5.75-5.87 (m, 1 H), 5.78 (d, J=2.4 Hz, 1 H), 5.93 (bt, 1 H), 6.41 (d, J=2.9 Hz, 1 H); ¹³C NMR (75 MHz, CDCl₃) δ 13.7, 22.3, 24.7, 28.8, 31.5, 35.9, 42.3, 52.4, 80.3, 116.9, 123.9, 133.5, 135.6; 168.4, 168.5. IR (NaCl) 2923, 1755, 1641, 1557 cm⁻¹. Anal. Calcd for C₁₅H₂₃NO₃: C, 67.9; H, 8.74; Found: C, 67.8; H, 8.67.

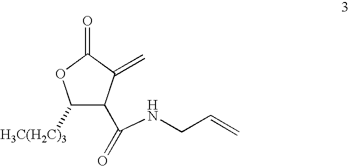

### (±)-α-Methylene-γ-butyrolactone-5-butyl-4-allyl amide (3)

From (±)-α-Methylene-γ-butyrolactone-5-butyl-4-carboxylic acid. (100 mg, 0.50 mmol) and allyl amine (41 μL, 0.55 mmol) following the above procedure was obtained 3 (68 mg, 57%) after flash chromatography (30-40% EtOAc/Hexanes). ¹H NMR (300 MH, CDCl₃) d 0.87 (t, J=6 Hz, 3 H), 1.28-1.50 (m, 4 H), 1.66-1.74 (m, 2 H), 3.41-3.45 (m, 1 H), 3.90 (app tt, J=5.7, 1.4 Hz, 2 H), 4.72-4.78 (m, 1 H), 5.14-5.20 (m, 2 H), 5.74-5.87 (m, 1 H), 5.78 (d, J=2.5 Hz, 1 H), 6.12 (bt, 1 H), 6.39 (d, J=2.8 Hz 1 H); ¹³C NMR (75 MHz, CDCl₃) δ 13.6, 22.2, 26.8, 35.5, 42.3, 52.5, 80.3, 117.0, 123.9, 133.5, 135.5, 168.3, 168.5. IR (NaCl) 2958, 1768, 1652, 1548. Anal. Calcd for C₁₃H₁₉NO₃: C, 65.8; H. 8.07; Found. C, 65.8; H, 8.07.

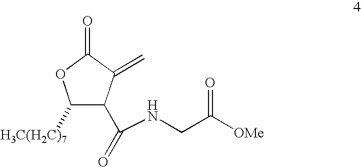

### (±)-α-Methylene-γ-butyrolactone-5-octyl-4-carboxy-methyl glycinate (4)

From C75 (39 mg, 0.15 mmol) and methyl glycinate hydrochloride (20 mg, 0.16 mmol) following the above procedure was obtained 4 (28 mg, 56%) after flash chromatography (35% EtOAc/Hexanes); mp. 94.5-95.5° C. ¹H NMR (300 MHz, CDCl₃) δ 0.85 (t, J=6.9 Hz, 3 H), 1.23 (s, 11 H), 1.41-1.49 (m, 1 H), 1.63-1.74 (m, 2 H), 3.46-3.49 (m, 1 H), 3.75 (s, 3 H), 3.97-4.14 (dd, J=5.4, 8 Hz, 2 H), 4.75 (dt, J=5.7, 7 Hz, 1 H), 5.88 (d, J=2 Hz, 1 H), 6.41 (d, J=2 Hz, 1 H), 6.55 (bs, 1 H);

¹³C NMR (75 MHz, CDCl₃) δ 14.1, 22.6, 24.8, 29.2, 29.2, 29.4, 31.8, 35.8, 41.4, 52.0, 52.6, 80.2, 124.8, 134.9, 168.6, 169.0, 169.9. IR (NaCl) 2915, 1768, 1737, 1644 cm⁻¹; Anal. Calcd for C₁₇H₂₇NO₅: C, 62.7; H, 8.36; Found: C, 62.7; H, 8.27.

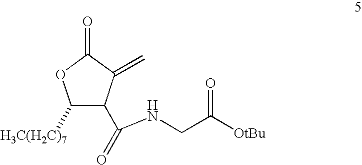

### (±)-α-Methylene-γ-butyrolactone-5-octyl-4-carboxy-tert-butyl-glycinate (5)

From C75 (100 mg, 0.39 mmol) and t-butyl glycinate hydrochloride (66 mg, 0.4 mmol) following the above procedure was obtained 5 (108 mg, 75%) after flash chromatography (35% Et2O-30% EtOAc/Hexanes). ¹H NMR (300 MHz, CDCl₃) δ 0.84 (t, J=6.8 Hz, 3 H), 1.25 (s, 12 H), 1.44 (s, 9 H), 1.65-1.73 (m, 2 H), 3.44-3.48 (m, 1 H), 3.92-3.95 (dd, J=3.6, 5 Hz, 2 H), 4.76 (dt, J=5.7, 7 Hz, 1 H), 5.88 (d, J=2 Hz, 1 H), 6.41 (d, J=2 Hz, 1 H), 4.47 (bt, 1 H). ¹³C NMR (75 MHz, CDCl₃) δ 13.9, 22.5, 24.8, 28.0, 29.1, 29.2, 29.3, 31.7, 35.8, 42.2, 51.9, 80.2, 82.6, 124.6, 135.1, 168.5, 168.6, 168.8. Anal. Calcd for C₂₀H₃₃NO₆: C, 65.4, H, 9.05; Found: C, 65.3; H. 9.02.

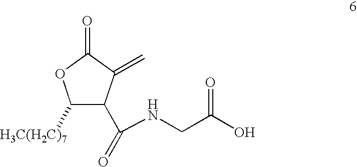

### (±)-α-Methylene-γ-butyrolactone-5-octyl-4-carboxy-glycinate (6)

From 5 (100 mg, 0.27 mmol) in CH₂Cl₂ (2.0 mL) was added TFA (1.3 mL) and the solution was allowed to stir for 3 h at rt. After evaporation of the solvents, column chromatography (50% EtOAc/2% CH₃CO₂H/Hexanes) provided pure 6 (61 mg, 73%). ¹H NMR (300 MHz, MeOD) δ 0.82 ( t, J=7 Hz, 3 H), 1.22 (s, 10 H), 1.28-1.38 (m, 2 H), 1.57-1.69 (m, 2 H), 3.55-3.59 (m, 2 H), 3.78-3.95 (ab-q, J=17 Hz, 2 H), 4.63 (qₐₚₚ, J=6.4 Hz, 1 H), 4.88 (bs, 1 H), 5.87 (d, J=2.6 Hz, 1 H), 6.19 (d, J=2.6 Hz, 1 H). ¹³C NMR (75 MHz, MeOD) δ 14.6, 23.8, 26.1, 30.5, 30.5, 30.6, 33.2, 36.6, 42.2, 52.8, 81.7, 124.8, 137.4, 170.8, 172.6, 172.5. IR (NaCl) 2915, 1769, 1731, 1644 cm⁻¹. Anal. Calcd for C₁₆H₂₅NO₅: C, 61.7; H, 8.09; Found: C, 61.7; H, 8.05.

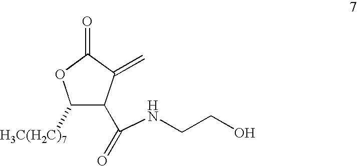

### (±)-α-Methylene-γ-butyrolactone-5-octyl-4-carboxylic acid ethanolamide (7)

From C75 (30 mg, 0.12 mmol) and ethanolamine (7.8 μl, 0.13 mmol) following the above procedure was obtained 7 (32 mg, 91%) after flash chromatography (50% EtOAc/Hexanes-100% EtOAc/2% CH₃CO₂H). ¹H NMR (300 MHz, CDCl₃) δ 0.86 (t, J=6.9 Hz, 3 H), 1.24 (s, 10 H), 1.35-1.48 (m, 2 H), 1.64-1.75 (m, 2 H), 3.40-3.57 (m, 3 H), 3.74 (t, J=5 Hz, 2 H), 4.73-4.79 (dt, J=5.7, 7 Hz, 1 H), 5.82 (d, J=2 Hz, 1 H); 6.42 (d, J=2 Hz, 1 H). 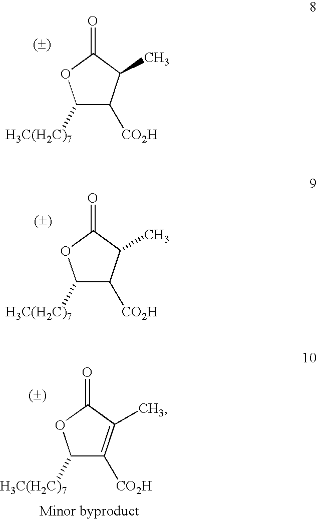
(8,9) To a solution of C75 (100 mg, 0.39 mmol) in EtOAc (3.0 mL) was added Pd (30 mg, 10% on Carbon) and H₂ (50 psi) for 2 h. The mixture was filtered-through celite and evaporated to give a mixture of diastereomers (1.8:1 for trans 9:cis 8). Column chromatography (20% EtOAc/2% CH₃CO₂H/Hexanes) yielded separate trans distereomer with unseparable isomerized byproduct (9:10, 3.8:1, 59.5 mg); and pure cis isomer (8, 32.7 mg,) (92% overall yield).

### (±)-α-Methyl-γ-butyrolactone-5-octyl-4-carboxylic acid (Trans diastereomer) (9)

¹H NMR (300 MHz, CDCl₃) δ 0.85 (t, J=7 Hz, 3 H), 1.23 (s, 10 H), 1.31 (d, J=7 Hz, 3 H), 1.41-1.50 (m, 2 H), 1.64-1.69 (m, 2 H), 2.62-2.69 (dd, J=9.6, 11.3 Hz, 1 H), 2.91-3.0 (dq, J=11.3, 7 Hz, 1 H), 4.42-4.49 (td, J=4, 9 Hz, 1 H). ¹³C NMR (75 MHz, CDCl₃) δ 13.9, 14.5, 22.6, 25.2, 29.1, 29.2, 29.3, 31.8, 32.7, 39.9, 53.9, 79.5, 176.0, 176.9. HRMS (ES) m/z calculated for C₁₄H₂₄O₄Na⁺ (M+Na⁺) 279.1566 observed. 279.1562.

### (±)-α-Methyl-γ-butyrolactone-5-octyl-4-carboxylic acid (Cis diastereomer) (8)

¹H NMR (300MHz, CDCl₃) δ 0.86 (t, J=6.9 Hz, 3 H), 1.25 (bs, 10 H), 1.29 (d, J=7.4 Hz, 3 H), 1.36-1.49 (m, 2 H), 1.63-1.71 (m, 2 H), 3.14 (dd, J=6, 9 Hz, 1 M), 3.02 (dq, J=7, 9 Hz, 1 H), 4.69 (qapp, J=6.3 Hz, 1 H). ¹³C NMR (75 MHz, CDCl₃) δ 11.8, 14.0, 22.6, 25.3, 29.1, 29.2, 29.3, 31.8, 34.7, 37.0, 49.9, 79.5, 175.4, 177.3. HRMS (ES) m/z calculated. For C₁₄H₂₄O₄Na⁺ (M+Na⁺) 279.1566 observed. 279.1568.

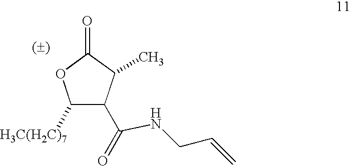

### (±)-α-Methyl-γ-butyrolactone-5-octyl-4-carboxylic acid allyl amide (11)

From 9 (52 mg, 0.20 mmol) and allyl amine (16 μl, 0.22 mmol) following the above procedure was obtained 11 (30 mg, 51%) after flash chromatography (40% Et₂O/Hexanes-30% EtOAc/Hexanes). ¹H NMR (300 MHz, CDCl₃) δ 0.86 (t, J=7 Hz, 3 H), 1.23-1.30 (m, 13 H), 1.38-1.49 (m, 2 H), 1.61-1.69 (m, 2 H), 2.29-2.36 (dd, J=9.3, 11.3 Hz, 1 H), 3.00-3.09 (dq, J=7, 11 Hz, 1 H), 3.92 (tt, J=1.5, 5.7 Hz, 2 H), 4.45-4.52 (m, 1 H), 5.15-5.22 (dd, J=10, 17 Hz, 2 H), 5.76-5.88 (m, 2 H). ¹³C NMR (75 MHz, CDCl₃) δ 13.9, 14.0, 22.6, 25.4, 29.1, 29.3, 29.3, 31.8, 34.7, 40.5, 42.2, 57.4, 80.4, 116.9, 133.5, 169.3, 177.4. HRMS (ES) m/z calculated for C₁₇H₂₉NO₃Na⁺ (M+Na⁺) 318.2039; observed. 318.2040.

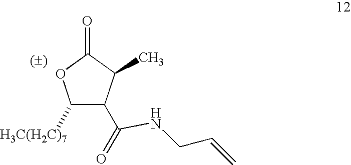

### (±)-α-Methyl-γ-butyrolactone-5-octyl-4-carboxylic acid allyl amide (12)

From 8 (32 mg, 0.12 mmol) and allylamine (10 μL, 0.13 mmol) following the above procedure was obtained 12 (20 mg, 53%) after flash chromatography (40% Et₂O/Hexanes-30% EtOAc/Hexanes). ¹H NMR (300 MHz, CDCl₃) δ 0.86 (t, J=7 Hz, 3 H), 1.21-1.25 (m, 13 H), 1.41-1.47 (m, 2 H), 1.58-1.67 (m, 2 H), 2.81-2.91 (m, 2 H), 3.83-3.96 (tt, J=1.5, 5 Hz, 2 H), 4.71-4.77 (m, 1 H), 5.13-5.21 (dd, J=10, 17Hz, 2 H), 5.75-5.87 (m, 2 H). ¹³C NMR (75 MHz, CDCl₃) δ 11.5, 14.0, 22.6, 25.4, 29.1, 29.2, 29.4, 31.8, 34.8, 37.4, 42.0, 51.2, 80.3, 116.9, 133.8, 169.1, 177.9. HRMS (ES) m/z calculated for C₁₇H₂₉NO₃Na⁺ (M+Na⁺) 318.2039; observed 318.2041.

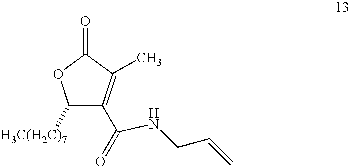

### 3-Methyl-5-octyl-5-oxo-2,5-dihydro-furan-3-carboxylic acid allylamide. (13)

From 3-Methyl-5-octyl-2-oxo-2,5-dihydro-furan-4-carboxylic acid (46 mg, 0.18 mmol) and allylamine (14 μl, 0.19 mmol) following the above procedure was obtained 13 (30 mg, 55%) after flash chromatography (40% EtOAc/Hexanes. ¹H NMR (300 MHz, CDCl₃) δ 0.85 (t, J=6.9 Hz, 3 ), 1.22 (s, 10 H), 1.46-1.55 (m, 2 H), 1.90-1.95 (m, 2 H), 2.04 (s, 3 H), 4.02 (td, J=1.4, 5.7 Hz, 2 H), 5.13-5.15 (m, 1 H), 5.18-5.25 (dd, J=10.6, 17.3 Hz, 2 H), 5.80-5.92 (ddt, J=10.3, 17, 5.7 Hz, 1 H). 6.07 (t, J=1.4 Hz, 1 H). ¹³C NMR (75 MHz, CDCl₃) δ 10.3, 14.0, 22.6, 24.8, 29.1, 29.2, 29.3, 31.8, 32.7, 42.0, 81.7, 117.5, 128.8, 133.1, 153.7, 162.1, 173.3. HEMS (ES) m/z calculated for C₁₇H₂₇NO₃Na⁺ (M+Na⁺) 316.1883 observed 316.1895.

### REFERENCES

1. Kunieda, T.; Nagamatsu, T.; Higuchi, T.; Hirobe, M. Tetrahedron Lett. 1988, 29, 2203-2206.
   Biological and Biochemical Methods
   Purification of FAS from ZR-75-1 Human Breast Cancer Cells.

Human FAS was purified from cultured ZR-75-1 human breast cancer cells obtained from the American Type Culture Collection. The procedure, adapted from Linn et al., 1981, and Kuhajda et al, 1994, utilizes hypotonic lysis, successive polyethyleneglycol (PEG) precipitations, and anion exchange chromatography. ZR-75-1 cells are cultured at 37° C. with 5% CO₂ in RPMI culture medium with 10% fetal bovine serum, penicillin and streptomycin.

Ten T150 flasks of confluent cells are lysed with 1.5 ml lysis buffer (20 mM Tris-HCl pH 7.5, 1 mM EDTA, 0.1 mM phenylmethanesulfonyl fluoride (PMSF), 0.1% Igepal CA-630) and dounce homogenized on ice for 20 strokes. The lysate is centrifuged in JA-20 rotor (Beckman) at 20,000 rpm for 30 minutes at 4° C. and the supernatant is brought to 42 ml with lysis buffer. A solution of 50% PEG 8000 in lysis buffer is added slowly to the supernatant to a final concentration of 7.5%. After rocking for 60 minutes at 4° C., the solution is centrifiged in JA-20 rotor (Beckman) at 15,000 rpm for 30 minutes at 4° C. Solid PEG 8000 is then added to the supernatant to a final concentration of 15%. After the rocking and centrifugation is repeated as above, the pellet is resuspended overnight at 4° C. in 10 ml of Buffer A (20 mM K₂HPO₄, pH 7.4). After 0.45 μM filtration, the protein solution is applied to a Mono Q 5/5 anion exchange column (Pharmacia). The column is washed for 15 minutes with buffer A at 1 ml/minute, and bound material is eluted with a linear 60-ml gradient over 60 minutes to 1 M KCl. FAS (MW˜270 kD) typically elutes at 0.25 M KCl in three 0.5 ml fractions identified using 4-15% SDS-PAGE with Coomassie G250 stain (Bio-Rad). FAS protein concentration is determined using the Coomassie Plus Protein Assay Reagent (Pierce) according to manufacturer's specifications using BSA as a standard This procedure results in substantially pure preparations of FAS (>95%) as judged by Coomassie-stained gels.

Measurement of FAS Enzymatic Activity and Determination of the IC₅₀ of the Compounds

FAS activity is measured by monitoring the malonyl-CoA dependent oxidation of NADPH spectrophotometrically at OD₃₄₀ in 96-well plates (Dils et al and Arslanian et at, 1975). Each well contains 2 μg purified FAS, 100 mM K₂HPO₄, pH 6.5, 1 mM dithiothreitol (Sigma), and 187.5 μM β-NADPH (Sigma). Stock solutions of inhibitors are prepared in DMSO at 2, 1, and 0.5 mg/ml resulting in final concentrations of 20, 10, and 5 μg/ml when 1 μl of stock is added per well. For each experiment, cerulenin (Sigma) is run as a positive control along with DMSO controls, inhibitors, and blanks (no FAS enzyme) all in duplicate.

The assay is performed on a Molecular Devices SpectraMax Plus Spectrophotometer. The plate containing FAS, buffers, inhibitors, and controls are placed in the spectrophotometer heated to 37° C. Using the kinetic protocol, the wells are blanked on duplicate wells containing 100 μl of 100 mM K₂HPO₄, pH 6.5 and the plate is read at OD₃₄₀ at 10 sec intervals for 5 minutes to measure any malonyl-CoA independent oxidation of NADPH. The plate is removed from the spectrophotometer and malonyl-CoA (67.4 μM, final concentration per well) and acetyl-CoA (61.8 μM, final concentration per well) are added to each well except to the blanks. The plate is read again as above with the kinetic protocol to measure the malonyl-CoA dependent NADPH oxidation. The difference between the Δ OD₃₄₀ for the malonyl-CoA dependent and non-malonyl-CoA dependent NADPH oxidation is the specific FAS activity. Because of the purity of the FAS preparation, non-malonyl-CoA dependent NADPH oxidation is negligible.

The IC₅₀ for the compounds against FAS is determined by plotting the Δ OD₃₄₀ for each inhibitor concentration tested, performing linear regression and computing the best-fit line, r² values, and 95% confidence intervals. The concentration of compound yielding 50% inhibition of FAS is the IC₅₀. Graphs of A OD₃₄₀ versus time are plotted by the SOFTmax PRO software (Molecular Devices) for each compound concentration. Computation of linear regression, best-fit line, r², and 95% confidence intervals are calculated using Prism Version 3.0 (Graph Pad Software).

Crystal Violet Cell Growth Assay

The crystal violet assay measures cell growth but not cytotoxicity. This assay employs crystal violet staining of fixed cells in 96-well plates with subsequent solubilization and measurement of OD₄₉₀ on a spectrophotometer. The OD₄₉₀ corresponds to cell growth per unit time measured. Cells are treated with the compounds of interest or vehicle controls and IC₅₀ for each compound is computed.

To measure the cytotoxicity of specific compounds against cancer cells, 5×10⁴ MCF-7 human breast cancer cells, obtained from the American Type Culture Collection are plated per well in 24 well plates in DMEM medium with 10% fetal bovine serum, penicillin, and streptomycin. Following overnight culture at 37° C. and 5% CO₂, the compounds to be tested, dissolved in DMSO, are added to the wells in 1 μl volume at the following concentrations: 50, 40, 30, 20, and 10 μg/ml in triplicate. Additional concentrations are tested if required. 1 μl of DMSO is added to triplicate wells as the vehicle control. C75 is run at 10, and 5 μg/ml in triplicate as positive controls.

After 72 hours of incubation, cells are stained with 0.5 ml of Crystal Violet stain (0.5% in 25% methanol) in each well. After 10 minutes, wells are rinsed, air dried, and then solubilized with 0.5 ml 10% sodium dodecylsulfate with shaking for 2 hours. Following transfer of 100 μl from each well to a 96-well plate, plates are read at OD₄₉₀ on a Molecular Devices SpectraMax Plus Spectrophotometer Average OD₄₉₀ values are computed using SOFTmax Pro Software (Molecular Devices) and IC₅₀ values are determined by linear regression analysis using Prism version 3.02 (Graph Pad Software, San Diego).

XTT Cytotoxicity Assay

The XTT assay is a non-radioactive alternative for the [⁵¹Cr] release cytotoxicity assay. XTT is a tetrazolium salt that is reduced to a formazan dye only by metabolically active, viable cells. The reduction of XTT is measured spectrophotometrically as OD₄₉₀-OD₆₅₀.

To measure the cytotoxicity of specific compounds against cancer cells, 9×10³ MCF-7 human breast cancer cells, obtained from the American Type Culture Collection are plated per well in 96 well plates in DMEM medium with 10% fetal bovine serum, msulin, penicillin, and streptomycin. Following overnight culture at 37° C. and 5% CO₂, the compounds to be tested, dissolved in DMSO, are added to the wells in 1 μl volume at the following concentrations: 80, 40, 20, 10, 5, 2.5, 1.25, and 0.625 μg/ml in triplicate. Additional concentrations are tested if required. 1 μl of DMSO is added to triplicate wells are the vehicle control. C75 is run at 40, 20, 10, 15, 12.5, 10, and 5 μg/ml in triplicate as positive controls.

After 72 hours of incubation, cells are incubated for 4 hours with the XTT reagent as per manufacturer's instructions (Cell Proliferation Kit II (XTT) Roche). Plates are read at OD₄₉₀ and OD₆₅₀ on a Molecular Devices SpectraMax Plus Spectrophotometer. Three wells containing the All reagent without cells serve as the plate blank. XTT data are reported as OD₄₉₀-OD₆₅₀. Averages and standard error of the mean are computed using SOFTmax Pro software (Molecular Dynamics).

The IC₅₀ for the compounds is defined as the concentration of drug leading to a 50% reduction in OD₄₉₀-OD₆₅₀ compared to controls. The OD₄₉₀-OD₆₅₀ are computed by the SOFTmax PRO software Molecular Devices) for each compound concentration. IC₅₀ is calculated by linear regression, plotting the FAS activity as percent of control versus drug concentrations. Linear regression, best-fit line, r², and 95% confidence intervals are determined using Prism Version 3.0 (Graph Pad Software).

Measurement of [¹⁴C]Acetate Incorporation into Total Lipids and Determination of IC₅₀ of Compounds

This assay measures the incorporation of [¹⁴C] acetate into total lipids and is a measure of fatty acid synthesis pathway activity in vitro. It is utilized to measure inhibition of fatty acid synthesis in vitro.

MCF-7 human breast cancer cells cultured as above, are plated at 5×10⁴ cells per well in 24-well plates. Following overnight incubation, the compounds to be tested, solubilized in DMSO, are added at 5, 10, and 20 μg/ml in triplicate, with lower concentrations tested if necessary. DMSO is added to triplicate wells for a vehicle control. C75 is run at 5 and 10 μg/ml in triplicate as positive controls. After 4 hours of incubation, 0.25 μCi of [¹⁴C]acetate (10 μl volume) is added to each well.

After 2 hours of additional incubation, medium is aspirated from the wells and 800 μl of chloroform:methanol (2:1) and 700 μl of 4 mM MgCl₂ is added to each well. Contents of each well are transferred to 1.5 ml Eppendorf tubes, and spun at full-speed for 2 minutes in a high-speed Eppendorf Microcentrifuge 5415D. After removal of the aqueous (upper) layer, an additional 700 μl of chloroform:methanol (2:1) and 500 μl of 4 mM MgCl₂ are added to each tube and then centrifuged for 1 minutes as above. The aqueous layer is removed with a Pasteur pipette and discarded. An additional 400 μl of chloroform:methanol (2:1) and 200 μl of 4 mM MgCl₂ are added to each tube, then centrifuged and aqueous layer is discarded. The lower (organic) phase is transferred into a scintillation vial and dried at 40° C. under N₂ gas. Once dried, 3 ml of scintillant (APB #NBC5104) is added and vials are counted for ¹⁴C. The Beckman Scintillation counter calculates the average cpm values for triplicates.

The IC₅₀ for the compounds is defined as the concentration of drug leading to a 50% reduction in [¹⁴C] acetate incorporation into lipids compared to controls. This is determined by plotting the average cpm for each inhibitor concentration tested, performing linear regression and computing the best-fit line, r² values, and 95% confidence intervals. The average cpm values are computed by the Beckman scintillation counter (model LS6500) for each compound concentration. Computation of linear regression, best-fit line, r², and 95% confidence intervals are calculated using Prism Version 3.0 (Graph Pad Software).

Carnitine Palmitoyltransferase-1 (CPT-1) Assay

CPT-1 catalyzes the ATP dependent transfer of long-chain fatty acids from acyl-CoA to acyl-carnitine that is inhibited by malonyl-CoA. As CPT-1 requires the mitochondrial membrane for activity, enzyme activity is measured in permeabilized cells or mitochondria This assay uses permeabilized cells to measure the transfer of [methyl-¹⁴C]L-carnitine to the organically soluble acyl-carnitine deriviative.

MCF-7 cells are plated in DMEM with 10% fetal bovine serum at 10⁶ cells in 24-well plates in triplicate for controls, drugs, and malonyl-CoA. Two hours before commencing the assay, drugs are added at the indicated concentrations made from stock solutions at 10 mg/ml in DMSO, vehicle controls consist of DMSO without drug. Since malonyl-CoA cannot enter intact cells, it is only added in the assay buffer to cells that have not been preincubated with drugs. Following overnight incubation at 37° C., the medium is removed and replaced with 700 μl of assay buffer consisting of: 50 mM imidazole, 70 mM KCl, 80 mM sucrose, 1 mM EGTA, 2 mM MgCl₂, 1 mM DTT, 1 mM KCN, 1 mM ATP, 0.1% fatty acid free bovine serum albumin, 70 μM palmitoyl-CoA, 0.25 μCi [methyl-¹⁴C]L-carnitine, 40 μg digitonin with drug, DMSO vehicle control, or 20 μM malonyl-CoA. The concentrations of drugs and DMSO in the assay buffer is the same as used in the 2 hr preincubation. After incubation for 6 minutes at 37° C., the reaction is stopped by the addition of 500 μl of ice-cold 4 M perchloric acid. Cells are then harvested and centrifuged at 13,000×g for 5 minutes. The pellet is washed with 500 μl ice cold 2 mM perchloric acid and centrifuged again. The resulting pellet is resuspended in 800 μl dH₂O and extracted with 150 μl of butanol. The butanol phase is counted by liquid scintillation and represents the acylcarnitine derivative.

Weight Loss Screen for Novel FAS Inhibitors

Balb/C mice (Jackson Labs) are utilized for the initial weight loss screening. Animals are housed in temperature and 12 hour day/night cycle rooms and fed mouse chow and water ad lib. Three mice are utilized for each compound tested with vehicle controls in triplicate per experiment. For the experiments, mice are housed separately for each compound tested three mice to a cage. Compounds are diluted in DMSO at 10 mg/ml when given at a dose of 30 mg/kg, and 30 mg/ml when given at a dose of 60 mg/kg, and mice are injected intraperitoneally with 60 mg/kg in approximately 100 μl of DMSO or with vehicle alone. Mice are observed and weighed daily; average weights and standard errors are computed with Excel (Microsoft). The experiment continues until treated animals reach their pretreatment weights. Select compounds are tested in animals housed in metabolic cages.

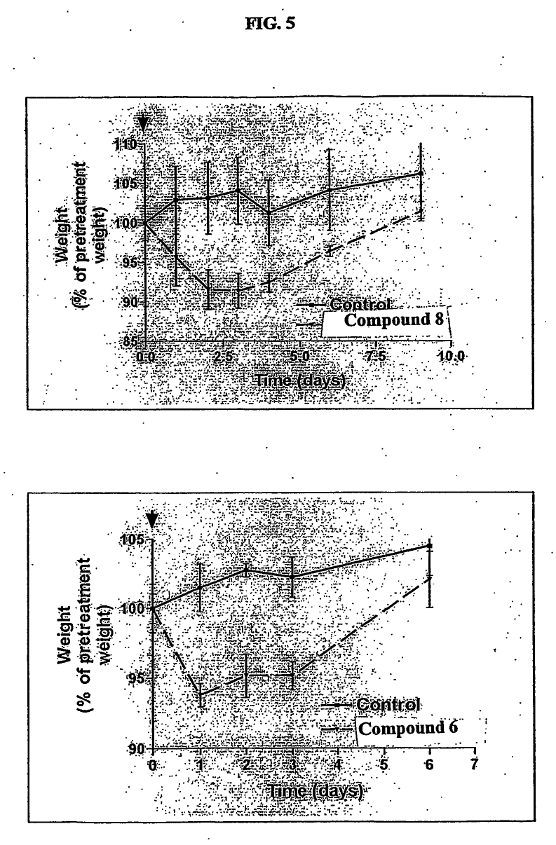

FIG. 5 shows the results of some in vivo testing for weight loss. Dosing of animals are identical to the screening experiments with three animals to a single metabolic cage. Animal weights, water and food consumption, and urine and feces production are measured daily. Three lean Balb/C mice (Harlan) maintained on mouse chow, are treated with compounds at doses indicated on day 0 or with vehicle (DMSO) control of equal volume. Compound 6 was solubilized in 40 μl DMSO while Compound 8 was solubilized in 60 μl DMSO. All were injected intraperitoneally. Weights were measured on days indicated. Error bars represent standard error of the mean.

Antimicrobial Properties

A broth microdilution assay is used to assess the antimicrobial activity of the compounds. Compounds are tested at twofold serial dilutions, and the concentration that inhibits visible growth (OD₆₀₀ at 10% of control) is defined as the MIC. Microorganisms tested include Staphylococcus aureus (ATCC #29213), Enterococcus faecalis (ATCC #29212), Pseudomonas aeruginosa (ATCC #27853), and Escherichia coli (ATCC #25922). The assay is performed in two growth media, Mueller Hinton Broth and Trypticase Soy Broth.

A blood (Tsoy/5% sheep blood) agar plate is inoculated from frozen stocks maintained in T soy broth containing 10% glycerol and incubated overnight at 37° C. Colonies are suspended in sterile broth so that the turbidity matches the turbidity of a 0.5 McFarland standard. The inoculum is diluted 1:10 in sterile broth (Mueller Hinton or Trypticase soy) and 195 ul is dispensed per well of a 96-well plate. The compounds to be tested, dissolved in DMSO, are added to the wells in 5 ul volume at the following concentrations: 25, 12.5, 6.25, 3.125, 1.56 and 0.78 ug/ml in duplicate. Additional concentrations are tested if required. 5 ul of DMSO added to duplicate wells are the vehicle control. Serial dilutions of positive control compounds, vancomycin (E. faecalis and S. aureus) and tobramycin (E. coli and P. aeruginosa), are included in each rum

After 24 hours of incubation at 37° C., plates are read at OD₆₀₀ on a Molecular Devices SpectraMax Plus Spectrophotometer. Average OD₆₀₀ values are computed using SOFTmax Pro Software (Molecular Devices) and MIC values are determined by linear regression analysis using Prism version 3.02 (Graph Pad Software, San Diego). The MIC is defined as the concentration of compound required to produce an OD₆₀₀ reading equivalent to 10% of the vehicle control reading.

In Vivo Testing for Anti-Tumor Activity

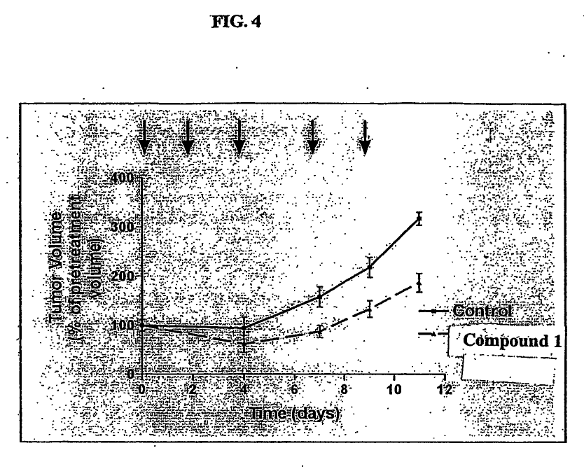

Subcutaneous flank xenografts of the human colon cancer cell line, HCT-116 in nu/nu female mice (Harlan) were used to study the anti-tumor effects of Compound 1 in vivo. All animal experiments complied with institutional animal care guidelines. 10⁷ HCT-116 cells (˜0.1 ml packed cells) were xenografted from culture in DMEM supplemented with 10% FBS into 20 athymic mice. Treatment began when measurable tumors developed about 3 days after inoculation. Compound 1 (10 mg/kg) was diluted into 40 μl DMSO and treated intraperitoneally (i.p.) 11 animals received JMM-III-231 10 mg/kg, i.p., at days indicated by arrows, and 11 received DMSO control. Tumors were measured on days indicated. One Compound 1 treated mouse died on day 10 from repeated i.p. injection. The results are shown in FIG. 4. Error bars represent standard error of the mean.

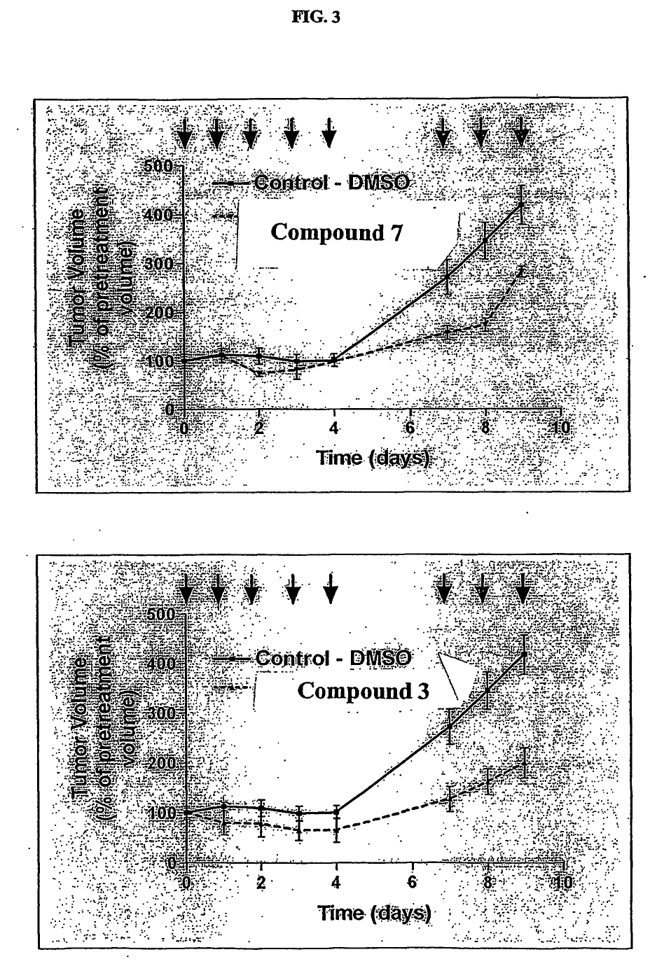

Subcutaneous flank xenografts of the human colon cancer cell line, HCT-116 in nu/nu female mice (Harlan) were used to study the anti-tumor effects of Compound 7 and Compound 3 in vivo. All animal experiments complied with institutional animal care guidelines. 10⁷ HCT-116 cells (˜0.1 ml packed cells) were xenografted from culture in DMEM supplemented with 10% FBS into 15 atymic mice. Treatment began when measurable tumors developed about 4 days after inoculation. Both Compound 7 and Compound 3 (10 mg/kg) were diluted into 20 μl DMSO for intraperitoneal (i.p.) injection. 5 animals received drugs i.p. at days indicated by arrows, and 5 received DMSO control. Tumors were measured on days indicated. The results are shown in FIG. 3. Error bars represent standard error of the mean.

Results of the Biological Testing

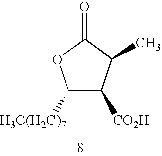

| FAS (IC₅₀) | ¹⁴C (IC₅₀) | XTT (IC₅₀) | Cr. Violet (IC₅₀) |
|---|---|---|---|
| Neg | 31 ug/ml | >80 ug/ml | >50 ug/ml |
| CPT I Stim | Weight Loss | | |
| Not Tested | 60 mg/kg: 8.6% (day 3); 30 mg/kg: 5.4% (day 2) | | |
| SA/MH (MIC) | SA/Tsoy (MIC) | PSAE/MH (MIC) | PSAE/Tsoy (MIC) |
| Not Tested | Not Tested | Not Tested | Not Tested |
| EF/MH (MIC) | EF/Tsoy (MIC) | Ecoli/MH (MIC) | Ecoli/Tsoy (MIC) |
| Not Tested | Not Tested | Not Tested | Not Tested |

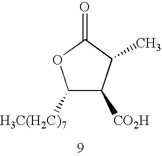

| FAS (IC₅₀) | ¹⁴C (IC₅₀) | XTT (IC₅₀) | Cr. Violet (IC₅₀) |
|---|---|---|---|
| Neg | Neg | >80 ug/ml | 49.0 ug/ml |
| CPT I Stim | Weight Loss | | |
| Not Tested | Not Tested | | |
| SA/MH (MIC) | SA/Tsoy (MIC) | PSAE/MH (MIC) | PSAE/Tsoy (MIC) |
| 43 ug/ml | 60 ug/ml | Neg | Neg |
| EF/MH (MIC) | EF/Tsoy (MIC) | Ecoli/MH (MIC) | Ecoli/Tsoy (MIC) |
| 109 ug/ml | 94 ug/ml | Neg | Neg |

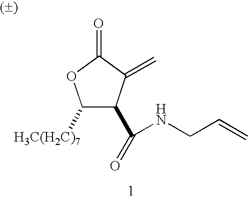

| FAS (IC₅₀) | ¹⁴C (IC₅₀) | XTT (IC₅₀) | Cr. Violet (IC₅₀) |
|---|---|---|---|
| Neg | 0.75 ± 0.4 ug/ml | 0.81 ± 0.01 ug/ml | 0.9 ± 0.5 ug/ml |
| CPT I Stim | Weight Loss | | |
| 400% of control/MCF | 60 mg/kg: 3 of 3 dead (day 3) 30 mg/kg: 10% (day 2) | | |
| at 10 ug.ml | 20 mg/kg: 11% (day 6); 10 mg/kg: 8.3% (day 7); | | |
| | 5 mg/kg: 4.6% (day 1) | | |
| SA/MH (MIC) | SA/Tsoy (MIC) | PSAE/MH (MIC) | PSAE/Tsoy (MIC) |
| 9.1 ± 1.9 ug ug/ml | 12.0 ± 0.5 ug/ml | Neg | Neg |
| EF/MH (MIC) | EF/Tsoy (MIC) | Ecoli/MH (MIC) | Ecoli/Tsoy (MIC) |
| 36 ug/ml | 25 ug/ml | Not Tested | Not Tested |

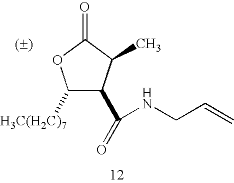

| FAS (IC₅₀) | ¹⁴C (IC₅₀) | XTT (IC₅₀) | Cr. Violet (IC₅₀) |
|---|---|---|---|
| Neg | 25.7 ug/ml | 59.4 ± 6.4 ug/ml | 43.9 ± 4.8 ug/ml |
| CPT I Stim | Weight Loss | | |
| Not Tested | 30 mg/kg: 2% (day 1) | | |
| SA/MH (MIC) | SA/Tsoy (MIC) | PSAE/MH (MIC) | PSAE/Tsoy (MIC) |
| 107 ug/ml | Neg | Neg | Neg |
| EF/MH (MIC) | EF/Tsoy (MIC) | Ecoli/MH (MIC) | Ecoli/Tsoy (MIC) |
| 91 ug/ml | 114 ug/ml | 108 ug/ml | Neg |

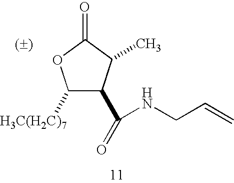

| FAS (IC₅₀) | ¹⁴C (IC₅₀) | XTT (IC₅₀) | Cr. Violet (IC₅₀) |
|---|---|---|---|
| 133 ug/ml | Neg | 20.8 ± 7.1 ug/ml | 30.0 ug/ml |
| CPT I Stim | Weight Loss | | |
| Not Tested | Not Tested | | |
| SA/MH (MIC) | SA/Tsoy (MIC) | PSAE/MH (MIC) | PSAE/Tsoy (MIC) |
| 80 ug/ml | 193 ug/ml | 218 ug/ml | 160 ug/ml |
| EF/MH (MIC) | EF/Tsoy (MIC) | Ecoli/MH (MIC) | Ecoli/Tsoy (MIC) |
| 84 ug/ml | Neg | Neg | 155 ug/ml |

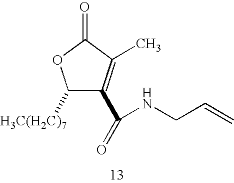

| FAS (IC₅₀) | ¹⁴C (IC₅₀) | XTT (IC₅₀) | Cr. Violet (IC₅₀) |
|---|---|---|---|
| 93 ug/ml | Neg (stim) | 27.8 ± 3.8 ug/ml | 23.7 ug/ml |
| CPT I Stim | Weight Loss | | |
| Not Tested | Not Tested | | |
| SA/MH (MIC) | SA/Tsoy (MIC) | PSAE/MH (MIC) | PSAE/Tsoy (MIC) |
| 79 ug/ml | 87 ug/ml | 280 ug/ml | 137 ug/ml |
| EF/MH (MIC) | EF/Tsoy (MIC) | Ecoli/MH (MIC) | Ecoli/Tsoy (MIC) |
| 115 ug/ml | 203 ug/ml | Neg | 199 ug/ml |

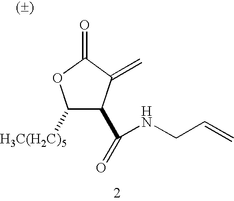

| FAS (IC₅₀) | ¹⁴C (IC₅₀) | XTT (IC₅₀) | Cr. Violet (IC₅₀) |
|---|---|---|---|
| 81 ug/ml | 3.3 ug/ml | 1.6 ± 0.1 ug/ml | 0.85 ± 0.08 ug/ml |
| CPT I Stim | Weight Loss | | |
| Not Tested | 30 mg/kg: 1 of 3 dead (day 1), 10 mg/kg: 6.7% (day 4) | | |
| SA/MH (MIC) | SA/Tsoy (MIC) | PSAE/MH (MIC) | PSAE/Tsoy (MIC) |
| 49 ug/ml | 47 ug/ml | Neg | Neg |
| EF/MH (MIC) | EF/Tsoy (MIC) | Ecoli/MH (MIC) | Ecoli/Tsoy (MIC) |
| 103 ug/ml | 38 ug/ml | Neg | Neg |

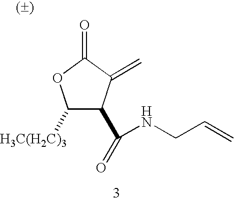

| FAS (IC₅₀) | ¹⁴C (IC₅₀) | XTT (IC₅₀) | Cr. Violet (IC₅₀) |
|---|---|---|---|
| 107 ug/ml | 1.8 ± 0.3 ug/ml | 2.4 ± 0.2 ug/ml | 2.2 ± 0.3 ug/ml |
| CPT I Stim | Weight Loss | | |
| Not Tested | 30 mg/kg: 3 of 3 dead (day 2); 10 mg/kg: 4.4% (day 4) | | |
| SA/MH (MIC) | SA/Tsoy (MIC) | PSAE/MH (MIC) | PSAE/Tsoy (MIC) |
| 65 ug/ml | 96 ug/ml | Neg | Neg |
| EF/MH (MIC) | EF/Tsoy (MIC) | Ecoli/MH (MIC) | Ecoli/Tsoy (MIC) |
| 190 ug/ml | 67 ug/ml | Neg | Neg |

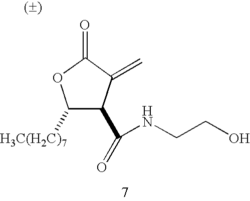

| FAS (IC₅₀) | ¹⁴C (IC₅₀) | XTT (IC₅₀) | Cr. Violet (IC₅₀) |
|---|---|---|---|
| Neg | 2.3 ± 1.3 ug/ml | 4.1 ± 2.2 ug/ml | 2.2 ± 1.0 ug/ml |
| CPT I Stim | Weight Loss | | |
| Not Tested | 30 mg/kg: 5.9% (day 2); 10 mg/kg: 1.7% (day 2) | | |
| SA/MH (MIC) | SA/Tsoy (MIC) | PSAE/MH (MIC) | PSAE/Tsoy (MIC) |
| 44 ug/ml | 48 ug/ml | Neg | Neg |
| EF/MH (MIC) | EF/Tsoy (MIC) | Ecoli/MH (MIC) | Ecoli/Tsoy (MIC) |
| Neg | 77 ug/ml | Neg | Neg |

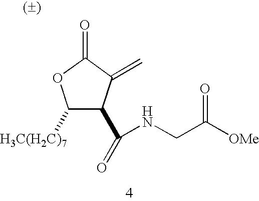

| FAS (IC₅₀) | ¹⁴C (IC₅₀) | XTT (IC₅₀) | Cr. Violet (IC₅₀) |
|---|---|---|---|
| Neg | 1.1 ± 0.03 ug/ml | 2.0 ± 0.5 ug/ml | 1.3 ± 0.09 ug/ml |
| CPT I Stim | Weight Loss | | |
| Not Tested | 30 mg/kg: 3 of 3 dead (day 1); 10 mg/kg: 3.1% (day 2) | | |
| SA/MH (MIC) | SA/Tsoy (MIC) | PSAE/MH (MIC) | PSAE/Tsoy (MIC) |
| 4.3 ug/ml | 26 ug/ml | Neg | Neg |
| EF/MH (MIC) | EF/Tsoy (MIC) | Ecoli/MH (MIC) | Ecoli/Tsoy (MIC) |
| 80 ug/ml | 245 ug/ml | Neg | 275 ug/ml |

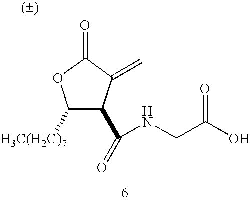

| FAS (IC₅₀) | ¹⁴C (IC₅₀) | XTT (IC₅₀) | Cr. Violet (IC₅₀) |
|---|---|---|---|
| Neg | | >80 ug/ml | >50 ug/ml |
| CPT I Stim | Weight Loss | | |
| Not Tested | 60 mg/kg: 9% (day 2), 1 death (day 3)/9.2% (day 2), | | |
| | 1 death (day 3) | | |
| | 30 mg/kg: 6.2% (day 1), 1 death (day 6)/10 mg/kg: | | |
| | 2.6% (day 2) | | |
| SA/MH (MIC) | SA/Tsoy (MIC) | PSAE/MH (MIC) | PSAE/Tsoy (MIC) |
| 72 ug/ml | 52 ug/ml | Neg | Neg |
| EF/MH (MIC) | EF/Tsoy (MIC) | Ecoli/MH (MIC) | Ecoli/Tsoy (MIC) |
| 219 ug/ml | 215 ug/ml | Neg | 235 ug/ml |

We claim:

1. Compounds of formula I: 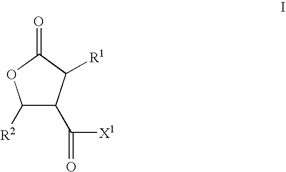
wherein
R¹═H, or C₁-C₂₀ alkyl, cycloalkyl, alkenyl, aryl, arylalkyl, or alkylaryl, ═CHR³, —C(O)OR³, —C(O)R³, —CH₂C(O)OR³, —CH₂C(O)NHR³, where R³ is H or C₁-C₁₀ alkyl, cycloalkyl, or alkenyl;
R²═C₁-C₂₀ alkyl, cycloalkyl, alkenyl, aryl, arylalkyl, or alkylaryl;
X¹═NHR⁴, where R⁴ is H, C₁-C₂₀ alkyl, cycloalkyl, alkenyl, aryl, arylalkyl, or alkylaryl, the R⁴ group optionally containing a carbonyl group, a carboxyl group, a carboxyamide group, an alcohol group, or an ether group, the R⁴ group further optionally containing one or more halogen atoms.

2. The compounds of claim 1, wherein R¹ is C₁-C₁₀ alkyl, cycloalkyl, alkenyl, aryl, arylalkyl, or alkylaryl, or ═CH₂.

3. The compounds of claim 2, wherein R¹ is CH₃ or ═CH₂.

4. The compounds of claim 3, wherein the compound is selected from the group consisting of:

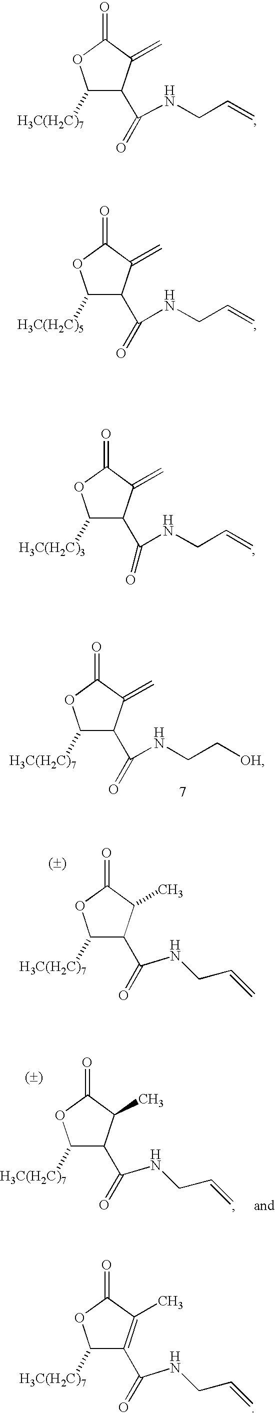

5. The compounds of claim 1, wherein R⁴ is —CH₂C(O)OR⁵ or —CH₂C(O)NHR⁵, where R⁵ is H, C₁-C₁₀ alkyl, cycloalkyl, alkenyl, aryl, arylalkyl, or alkylaryl.

6. The compounds of claim 5, wherein the compound is selected from the group consisting of:

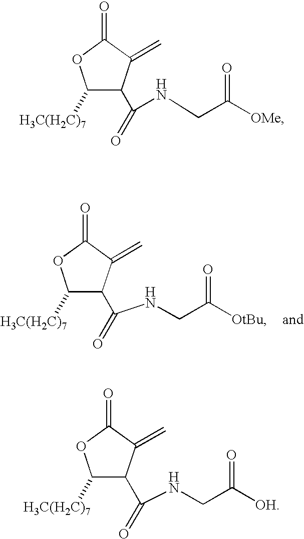

7. Compounds of formula II: 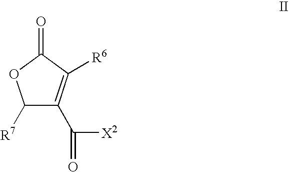
wherein
R⁶═H, or C₁-C₂₀ alkyl, cycloalkyl, alkenyl, aryl, arylalkyl, or alkylaryl, —C(O)OR⁸, —C(O)R⁸, —CH₂C(O)OR⁸, —CH₂C(O)NHR⁸, where R⁸ is H or C₁-C₁₀ alkyl, cycloalkyl, or alkenyl;
R⁷═C₁-C₂₀ alkyl, cycloalkyl, alkenyl, aryl, arylalkyl, or alkylaryl;
X²═NHR⁹, where R⁹ is H, C₁-C₂₀ alkyl, cycloalkyl, alkenyl, aryl, arylalkyl, or alkylaryl, the R⁹ group optionally containing a carbonyl group, a carboxyl group, a carboxyamide group, an alcohol group, or ah ether group, the R⁹ group further optionally containing one or more halogen atoms;
with the proviso that when R⁶ is —CH₃, and R⁷ is n-C₁₃H₂₇, X² is not —NHC₂H₅.

8. The compounds of claim 7, wherein R⁶ is C₁-C₁₀ alkyl, cycloalkyl, alkenyl, aryl, arylalkyl, or alkylaryl.

9. The compounds of claim 8, wherein R⁶ is —CH₃.

10. The compounds of claim 7, wherein R⁹ is —CH₂C(O)OR¹⁰ or —CH₂C(O)NHR¹⁰, where R¹⁰ is H, C₁-C₁₀ alkyl, cycloalkyl, alkenyl, aryl, arylalkyl, or alkylaryl.

11. Compounds of formula IV: 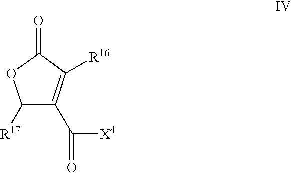
wherein
R¹⁶═H, or C₁-C₂₀ alkyl, cycloalkyl, alkenyl, aryl, arylalkyl, or alkylaryl, —C(O)OR¹⁸, —C(O)R¹⁸, —CH₂C(O)OR¹⁸, —CH₂C(O)NHR¹⁸, where R¹⁸ is H or C₁-C₁₀ alkyl, cycloalkyl, or alkenyl;
R¹⁷═C₁-C₂₀ alkyl, cycloalkyl, alkenyl, aryl, arylalkyl, or alkylaryl;
X⁴═OR¹⁹, where R¹⁹ is C₁-C₂₀ alkyl, cycloalkyl, alkenyl, aryl, arylalkyl, or alkylaryl, the R¹⁹ group optionally containing a carbonyl group, a carboxyl group, a carboxyamide group, an alcohol group, or an ether group, the R¹⁹ group further optionally containing one or more halogen atoms;
with the proviso that when R¹⁶ is —CH₃ and R¹⁹ is —CH₃, then R¹⁷ is not substituted or unsubstituted phenyl, -nC₃H₇, -nC₅H₁₁, -nC₁₃H₂₇,
and with the further proviso that when R¹⁶ is H and R¹⁹ is —CH₃, then R¹⁷ is not substituted or unsubstuted phenyl or —CH₃, and when R¹⁶ is H and R¹⁹ is —CH₂CH₃, then R¹⁷ is not -iC₃H₇, or substituted or unsubstituted phenyl.

12. The compounds of claim 11, wherein R¹⁶ is C₁-C₁₀ alkyl, cycloalkyl, alkenyl, aryl, arylalkyl, or alkylaryl.

13. The compounds of claim 12, wherein R¹⁶ is —CH₃.

14. The compounds of claim 11, wherein R¹⁹ is —CH₂C(O)OR²⁰ or —CH₂C(O)NHR²⁰, where R²⁰ is C₁-C₁₀ alkyl, cycloalkyl, alkenyl, aryl, arylalkyl, or alkylaryl.

15. Compounds of formula V: 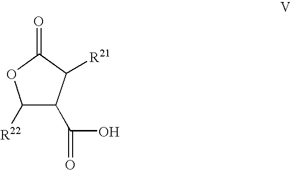
wherein
R²¹═C₂-C₂₀ alkyl, cycloalkyl, alkenyl, aryl, arylalkyl, or alkylaryl, ═CHR²³, —C(O)OR²³, —C(O)R²³, —CH₂C(O)OR²³, —CH₂C(O)NHR²³, where R²³ is H or C₁-C₁₀ alkyl, cycloalkyl, or alkenyl, except when R²¹ is ═CHR²³, R²³ is not H;
R²²═C₁-C₂₀ alkyl, cycloalkyl, alkenyl, aryl, arylalkyl, or alkylaryl;
with the proviso that when R²¹ is —COOH, then R²² is not —CH₃, -nC₅H₁₁, or C₁₃H₂₇, and with the further proviso that when R²¹ is —CH₂COOH, then R²² is not —CH₃, —CH₂CH₃, or -iC₅H₁₁.

16. The compounds of claim 15, wherein R²¹ is C₂-C₁₀ alkyl, cycloalkyl, alkenyl, aryl, arylalkyl, or alkylaryl.

17. The compounds of claim 16, wherein R²¹ is ═CH₂.

18. Compounds of formula VI: 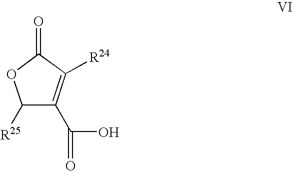
wherein:
R²⁴═C₂-C₂₀ alkyl, cycloalkyl, alkenyl, aryl, arylalkyl, or alkylaryl, —C(O)OR²⁶, —C(O)R²⁶, —CH₂C(O)OR²⁶, —CH₂C(O)NHR²⁶, where R²⁶ is H or C₁-C₁₀ alkyl, cycloalkyl, or alkenyl;
R²⁵═C₁-C₂₀ alkyl, cycloalkyl, alkenyl, aryl, arylalkyl, or alkylaryl;
with the proviso that when R²⁴ is —COOH, then R²⁵ is not —CH₃, -nC₅H₁₁, or C₁₃H₂₇, and with the further proviso that when R²⁴ is —CH₂COOH, then R²⁵ is not CH₃, —CH₂CH₃, or

19. The compounds of claim 18, wherein R²¹ is C₂-C₁₀ alkyl, cycloalkyl, alkenyl, aryl, arylalkyl, or alkylaryl.

20. Compounds of formula VII: 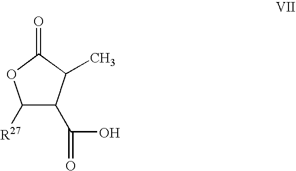
wherein R²⁷═C₃-C₄ alkyl, C₆-C₁₀ alkyl, C₁₂ alkyl, C₁₄ alkyl, C₁₆-C₂₀ alkyl.

21. The compounds of claim 20, selected from the group consisting of:

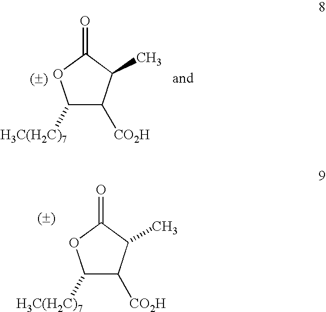

22. A compound of formula VIII: 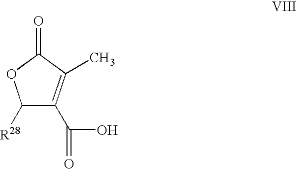
wherein R²⁸ is C₁-C₂₀ alkyl, cycloalkyl, alkenyl, aryl, arylalkyl, or alkylaryl, with the proviso that R²⁸ is not —CH₃, -nC₃H₇, -nC₁₁H₂₃, or -nC₁₃H₂₇.

23. A pharmaceutical composition comprising a pharmaceutical diluent and a compound of formula IX: 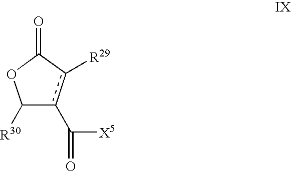
R²⁹═H, or C₁-C₂₀ alkyl, cycloalkyl, alkenyl, aryl, arylalkyl, or alkylaryl, ═CHR³¹, —C(O)OR³¹, —C(O)R³¹, —CH₂C(O)OR³¹, —CH₂C(O)NHR³¹, where R³¹ is H or C₁-C₁₀ alkyl, cycloalkyl, or alkenyl;
R³⁰═C₁-C₂₀ alkyl, cycloalkyl, alkenyl, aryl, arylalkyl, or alkylaryl;
X⁵═—OR³², or —NHR³², where R³² is H, C₁-C₂₀ alkyl, cycloalkyl, alkenyl, aryl, arylalkyl, or alkylaryl, the R³² group optionally containing a carbonyl group, a carboxyl group, a carboxyamide group, an alcohol group, or an ether group, the R³² group further optionally containing one or more halogen atoms;
with the proviso that when R²⁹ is ═CH₂, then X⁵ is not OH.

24. The pharmaceutical compositions of claim 23, wherein R²⁹ is C₁-C₁₀ alkyl, cycloalkyl, alkenyl, aryl, arylalkyl, or alkylaryl, or ═CH₂.

25. The pharmaceutical compositions of claim 24, wherein R²⁹ is —CH₃ or ═CH₂.

26. The pharmaceutical compositions of claim 23, wherein R³² is —CH₂C(O)OR³³ or —CH₂C(O)NH³³, where R³³ is C₁-C₁₀ alkyl, cycloalkyl, alkenyl, aryl, arylalkyl, or alkylaryl.

27. The pharmaceutical compositions of claim 23, where R²⁹ is —C₆H₁₃ or —C₈H₁₇.

28. The pharmaceutical compositions of claim 23, wherein the compound is selected from the group consisting of:

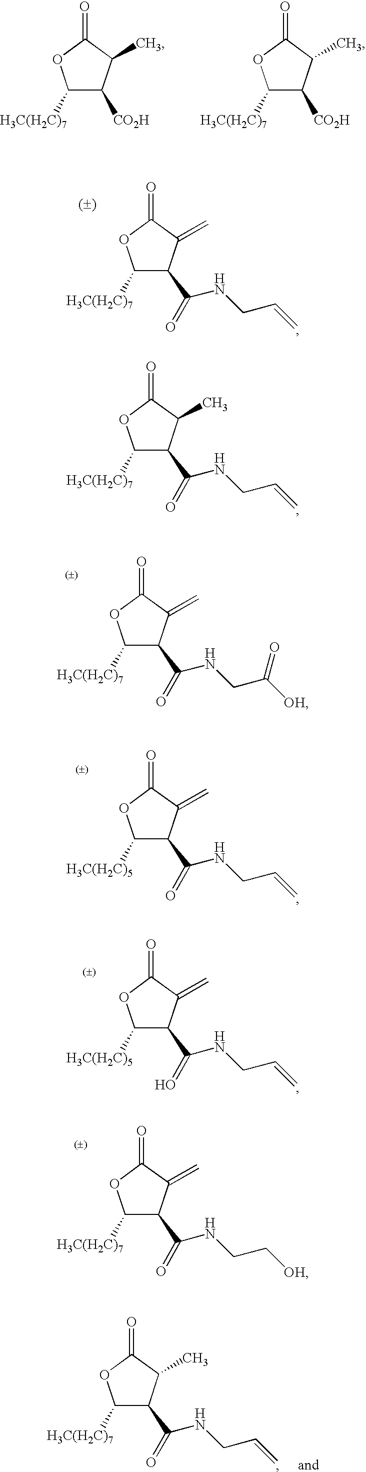

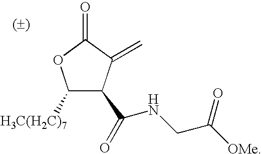

29. A pharmaceutical composition comprising a pharmaceutical diluent and a compound according to claim 1.

30. A pharmaceutical composition comprising a pharmaceutical diluent and a compound according to claim 7.

31. A pharmaceutical composition comprising a pharmaceutical diluent and a compound according to claim 11.

32. A pharmaceutical composition comprising a pharmaceutical diluent and a compound according to claim 15.

33. A pharmaceutical composition comprising a pharmaceutical diluent and a compound according to claim 18.

34. A pharmaceutical composition comprising a pharmaceutical diluent and a compound according to claim 20.

35. A pharmaceutical composition comprising a pharmaceutical diluent and a compound according to claim 22.

36. A pharmaceutical composition comprising a pharmaceutical diluent and a compound according to Formula III. 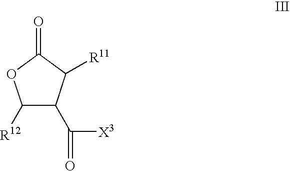
wherein
R¹¹═H, or C₁-C₂₀ alkyl, cycloalkyl, alkenyl, aryl, arylalkyl, or alkylaryl, ═CHR¹³, —C(O)OR¹³, —C(O)R¹³, —CH₂C(O)OR¹³, —CH₂C(O)NHR¹³, where R¹³ is H or C₁-C₁₀ alkyl, cycloalkyl, or alkenyl;
R¹²═C₁-C₂₀ alkyl cycloalkyl, alkenyl, aryl, arylalkyl, or alkylaryl;
X³═OR¹⁴, where R¹⁴ is C₁-C₂₀ alkyl, cycloalkyl, alkenyl, aryl, arylalkyl, or alkylaryl, the R¹⁴ group optionally containing a carbonyl group, a carboxyl group, a carboxyamide group, an alcohol group, or an ether group, the R¹⁴ group further optionally containing one or more halogen atoms.

37. The pharmaceutical formulation of claim 36, wherein R¹¹ is C₁-C₁₀ alkyl, cycloalkyl, alkenyl, aryl, arylalkyl, or alkylaryl, or ═CH₂.

38. The pharmaceutical formulation of claim 37, wherein R¹¹ is —CH₃ or ═CH₂.

39. The pharmaceutical formulation of claim 36, wherein R¹⁴ is —CH₂C(O)OR¹⁵ or —CH₂C(O)NHR¹⁵, where R¹⁵ is C₁-C₁₀ alkyl, cycloalkyl, alkenyl, aryl, arylalkyl, or alkylaryl.

40. A method of inducing weight loss in an animal or human subject comprising administering an effective amount of a pharmaceutical composition according to claim 23 to said subject.

41. The method of claim 40, wherein the subject is a human.

42. The method of claim 40, wherein the subject is an animal.

43. The method of claim 41, wherein the pharmaceutical composition comprises a compound selected from the group consisting of:

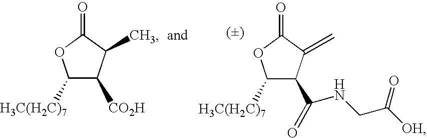

44. The method of claim 42, wherein the pharmaceutical composition comprises a compound selected from the group consisting of:

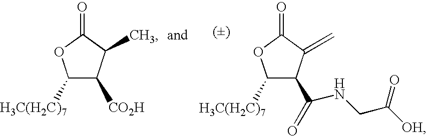

45. A method of inhibiting growth of cancer cells in an animal or human subject, comprising administering an effective amount of a pharmaceutical composition according to claim 23 to said subject.

46. The method of claim 45, wherein the subject is a human.

47. The method of claim 45, wherein the subject is an animal.

48. The method of claim 46, wherein the pharmaceutical composition comprises a compound selected from the group consisting of:

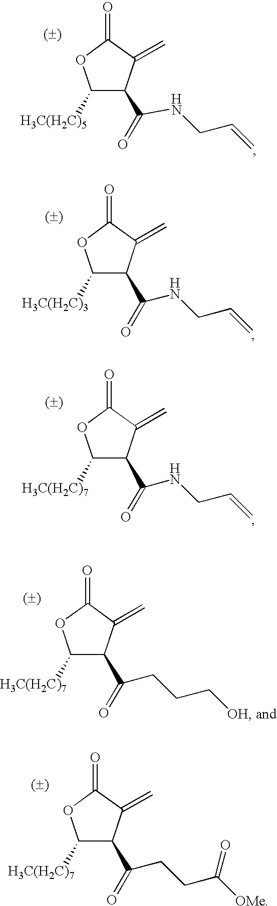

49. The method of claim 47, wherein the pharmaceutical composition comprises a compound selected from the group consisting of:

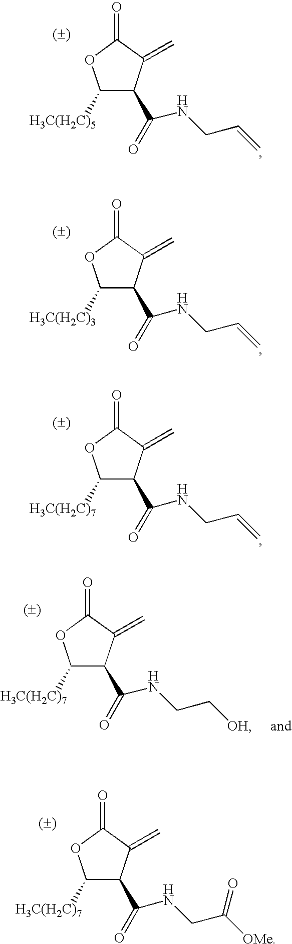

50. A method of stimulating the activity of CPT-1 in an animal or human subject comprising administering an effective amount of a pharmaceutical composition according to claim 23 to said subject.

51. The method of claim 50, wherein the subject is a human.

52. The method of claim 50, wherein the subject is an animal.

53. The method of claim 51, wherein the compound is:

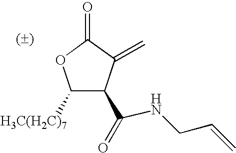

54. The method of claim 52, wherein the compound is:

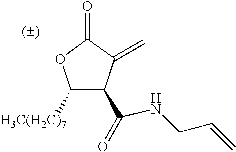

55. A method of inhibiting the activity of neuropeptide-Y in an animal or human subject comprising administering an effective amount of a pharmaceutical composition according to claim 23 to said subject.

56. The method of claim 55, wherein the subject is a human.

57. The method of claim 55, wherein the subject is an animal.

58. A method of inhibiting fatty acid synthase activity in an animal or human subject comprising administering an effective amount of a pharmaceutical composition according to claim 23 to said subject.

59. The method of claim 58, wherein the subject is a human.

60. The method of claim 58, wherein the subject is an animal.

61. The method of claim 59, wherein the compound is selected from the group consisting of:

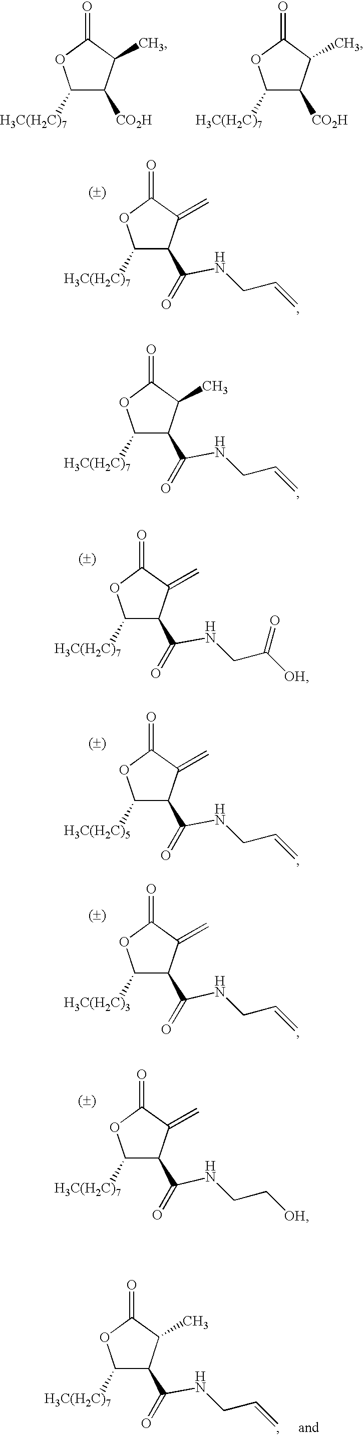

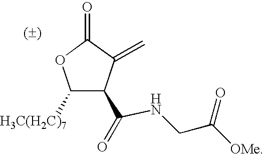

62. The method of claim 60, wherein the compound is selected from the group consisting of:

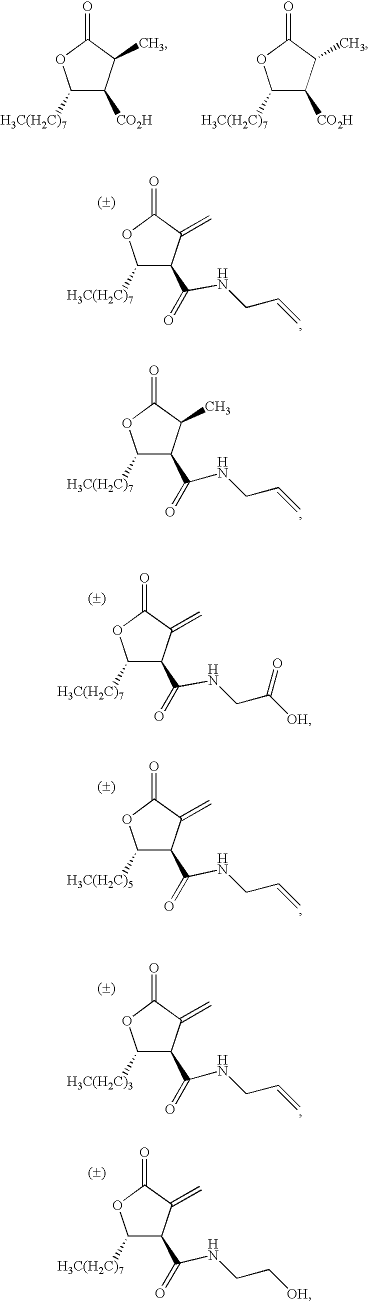

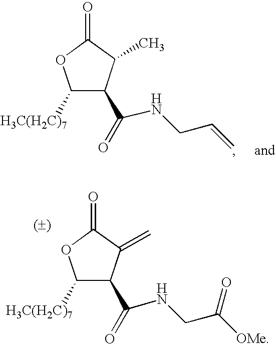

63. A method of inhibiting growth of invasive microbial cells in an animal or human subject comprising the administration of an effective amount of a pharmaceutical composition according to claim 23 to said subject.

64. The method of claim 63, wherein the subject is a human.

65. The method of claim 63, wherein the subject is an animal.

66. The method of claim 64, wherein the compound is selected from the group consisting of:

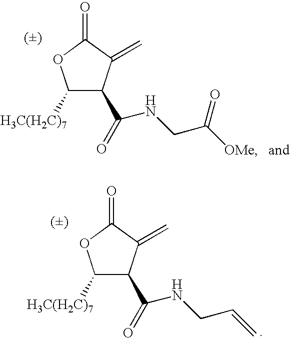

67. The method of claim 65, wherein the compound is selected from the group consisting of: [chemical structure]
